(19) United States
(12) Patent Application Publication
(10) Pub. No.: US 2023/0197204 A1
(43) Pub. Date: Jun. 22, 2023

(54) LOCAL-ANCESTRY INFERENCE WITH MACHINE LEARNING MODEL

(71) Applicants: Chan Zuckerberg Biohub, Inc., San Francisco, CA (US); The Board of Trustees of the Leland Stanford Junior University, Stanford, CA (US)

(72) Inventors: Daniel Mas Montserrat, Stanford, CA (US); Alexander Ioannidis, Stanford, CA (US); Arvind Kumar, Stanford, CA (US); Carlos Bustamante, Berkeley, CA (US); Richa Rastogi, Stanford, CA (US); Helgi Hilmarsson, Stanford, CA (US)

(21) Appl. No.: 17/996,183

(22) PCT Filed: Apr. 15, 2021
(86) PCT No.: PCT/US2021/027478

§ 371 Date: Oct. 13, 2022

Related U.S. Application Data
(60) Provisional application No. 63/010,467, filed on Apr. 15, 2020.

(51) Int. Cl. G16B 40/20; G16B 20/20; G06N 20/20

(52) U.S. Cl. CPC G16B 40/20; G16B 20/20; G06N 20/20

(57) ABSTRACT

A computer-implemented method comprises: storing a trained machine learning model, the machine learning model comprising a predictor sub-model and a smoothing sub-model, the machine learning model being trained based on segments of training genomic sequences that have known ancestral origins; receiving data representing an input genomic sequence of the subject, the input genomic sequence covering a plurality of segments including a plurality of single nucleotide polymorplasms (SNP) sites of the genome of the subject, wherein each segment comprises a sequence of SNP values at the SNP sites, each SNP value specifying a variant at the SNP site; determining, using the predictor sub-model and based on the data, an initial ancestral origin estimate of each segment of SNP values; and performing, by the smoothing sub-model for each segment, a smoothing operation over the initial ancestral origin estimates to obtain a final prediction result for the ancestral origin of the segment.

### RELATED APPLICATION

This patent application claims priority to U.S. Provisional Patent Application Ser. No. 63/010,467, filed Apr. 15, 2020, entitled “LOCAL-ANCESTRY INFERENCE WITH MACHINE LEARNING MODEL,” which is assigned to the assignees thereof and is incorporated herein by reference in its entirety for all purposes.

This invention was made with Government support under grant number HG009080 awarded by the National Institutes of Health. The Government has certain rights in the invention.

### BACKGROUND

Although most sites in a deoxyribonucleic acid (DNA) sequence do not vary between individuals, about two percent (5 million positions) do. These are referred to as single nucleotide polymorphisms (SNPs). Modern human populations, originating from different continents and different subcontinental regions, exhibit discernible differences in the frequencies of SNP variants at each site in the DNA sequence in their genomes. Because DNA is inherited as an intact sequence with only rare, random swaps in ancestry (between the two parental DNA sequences) at each generation, ancestral SNPs form contiguous segments allowing for powerful ancestry inference based on patterns of contiguous SNP variants.

Local-ancestry inference uses the pattern of variation observed at various sites along an individual's genome to estimate the ancestral origin of an individual's DNA. The ability to accurately infer the ancestry for each segment of an individual's DNA, at milliMorgan resolution, is important to disentangle the role of genetics and environment for complex traits including illness predisposition, since populations with a common ancestry share complex physical and medical traits. For example, Puerto Ricans living in the United States have the highest mortality of asthma and Mexicans have the lowest. Elucidating the genetic associations within populations for biomedical traits (like height, blood pressure, cholesterol levels, and predisposition to certain illness) can inform the development of treatments, and allow for the building of predictors of disease risk, known as polygenic risk scores. However, because the correlations between neighboring genetic variants are ancestry dependent, applying these risk scores to an individual's genome requires knowledge of the individual's ancestry at each site along the genome. With the increasing diversity of admixed modern cosmopolitan populations, such ancestry-specific analysis along the genome is becoming an increasingly complex and important computational problem.

Accordingly, it is desirable for new techniques to estimate the ancestral origin(s) of segments of genetic variants (e.g., SNP) in a DNA sequence.

### BRIEF SUMMARY

Embodiments of the present disclosure provide methods, systems, and apparatus for estimating the ancestral origin(s) of segments of genetic variants (e.g., SNP) in a DNA sequence using a machine learning model. The machine learning model can process data representing a haploid or diploid DNA sequence obtained from, for example, a genome sequencing operation that provides a genomic sequence of the subject, a DNA microarray that contains segments of DNAs, etc. The machine learning model can generate predictions of ancestral origins for segments of SNPs in a genome (e.g., mapped to different regions in a reference genome) at a high resolution, such as at milliMorgan resolution.

According to some embodiments, the machine learning model comprises a predictor sub-model to generate the initial ancestral origin estimates of segments of SNPs, and a smoothing sub-model to perform smoothing operations over the initial estimates. The smoothing sub-model can perform smoothing operations to remove or reduce discontinuities in the initial ancestral origin estimates. In some examples, the predictor sub-model can be configured as a classifier to classify segments of SNPs into one of a set of candidate ancestral origin categories (e.g., East Asia, South Asia, Middle East, Africa, Europe, Polynesia, Oceania, etc.) based on a classification operation. In some examples, the predictor sub-model can be configured as a regressor to estimate geographical coordinates (such as latitude and longitude) of ancestral origin locales of the segments of SNPs based on a regression operation. The geographical coordinates of an ancestral origin locale can pin point a much higher resolution, and continuously varying, set of geographical locations than a finite set of candidate ancestral origin categories. For example, the geographical coordinates can refer to any location (e.g., Oxford) within a particular country (e.g., Britain), whereas the ancestral origin category can refer to only a finite set of locations, typically a continent (e.g., Africa) or a sub-continent (e.g., North Africa) or a country (e.g., Japan). Moreover, in some cases, a regressor can provide useful ancestral estimates even for closely related populations, which can present problems for a classifier that treats each ancestry misclassification equally even though some ancestries are much more related than others. In some examples, the machine learning model can be trained to generate coordinates representing an ancestral origin/breed in a multi-dimensional space having dimensions obtained from a dimensionality-reduction.

The predictor sub-model and smoothing sub-model can include various topologies, such as a neural network model, a gradient boosting model, etc. In one example, the predictor sub-model can include one or more fully-connected neural networks, each assigned to process a segment of SNPs in an input DNA sequence to generate an initial ancestral origin estimate of the segment. The initial ancestral origin estimate may include, for example, a probability of the segment of SNPs belonging to a particular ancestral origin category, an estimate of geographical coordinates of an ancestral origin locale, etc. The smoothing sub-model can include a convolutional neural network to, as part of the smoothing operation, convolve a kernel with a set of neighboring initial ancestral origin estimates to generate a smoothed version of the initial estimates as final predictions. In some examples, the predictor sub-model can include a plurality of fully-connected neural networks, with each network having a different set of weights trained for a different set of SNP sites to process an SNP segment. In some examples, the predictor sub-model can include a single fully-connected neural network having a single set of weights to process different SNP segment. The single fully-connected neural network also accepts a segment index associated with each SNP segment, which allows the neural network to process different SNP segments differently using the same set of weights.

In another example, the predictor sub-model and the smoothing sub-model can include a plurality of decision tree models. The decision tree models in the predictor sub-model can generate decision outputs for a segment of SNPs. The outputs of the decision tree models can be combined to generate an initial ancestral origin estimate. The decision trees in the smoothing sub-model can generate decision outputs based on subsets of initial ancestral origin estimates, and the decision outputs can be combined to provide a smoothed version of the initial estimates as final predictions. In some examples, the predictor sub-model can include a plurality of decision tree models, with each decision tree model having a different set of tree parameters (e.g., different topologies, different decision criteria, etc.) trained for a different set of SNP sites to process an SNP segment. In some examples, the predictor sub-model can include a single decision tree model having a single set of tree parameters (e.g., a single topology, a single set of decision criteria, etc.) to process different SNP segment. The single decision tree model also accepts a segment index associated with each SNP segment, which allows the single decision tree model to process different SNP segments differently using the same set of tree parameters.

The machine learning model can be trained using various techniques. For example, in a case where the machine learning model comprises neural network models, the machine learning model can be trained based on minimizing a loss function that compares predictions of ancestral origins output by the machine learning model and true ancestral origins of segments of training SNPs sequences. A loss gradient can be generated from the loss function, and the loss gradient can be used to update the weights of the fully-connected neural network of the predictor sub-model, as well as the kernel of the convolutional neural network of the smoothing sub-model. In a case where the machine learning model comprises decision trees, the decision trees of the predictor sub-model and the smoothing sub-model can be trained separately based on a gradient boosting operation, which adds new decision trees sequentially based on a result of adjusting the decisions of preceding decision trees to better fit the known ancestral origin categories and/or known geographical coordinates of ancestral origin locales of segments of training SNPs sequences.

The machine learning model can be trained based on training data derived from full genome data of population of known ancestral origins, including individuals from various locales of Africa, East Asia, and Europe, as well as smaller geographic regions. From the full genomic sequence of these individuals, simulated genomic sequences of simulated admixed descendants of these individuals can be generated based on a forward simulation (e.g., Wright-Fisher) over a series of generations. A set of training data comprising genomic sequences of simulated admixed descendants of these individuals (e.g., over numerous generations), as well as the known ancestral origins of SNP segments of the simulated genomic sequences, can be used to train and validate the machine learning model. The training allows the machine learning model to learn from the relationships between patterns of SNP variants mapped to different DNA sites and their ancestral origins to perform local-ancestry inference. In a case where the predictor model includes a single neural network model or a single decision tree model, the model can be trained based on inputs including the segment indices to allow the single model to adjust the weights to account for different SNP sites.

With the disclosed embodiments, a machine learning model can be trained to identify ancestry-specific patterns of sequences of SNPs with a high resolution (e.g., for segments of SNPs at milliMorgan resolution). By training the machine learning model with training data including genomic sequences of many simulated admixed descendants of these individuals, the machine learning model can become robust to populations and individuals having different admixture histories. The robustness of the machine learning model can be improved when the model is trained as a regressor to estimate geographical coordinates of ancestral origin locales of the segments of SNPs based on a regression operation, which can provide useful ancestral estimates even for closely related populations. The robustness of the machine learning model can be further improved by the smoothing sub-model, which can not only removes discontinuities in the initial ancestral origin estimates, but can be also trained by the training data to remove the discontinuities.

In addition, the machine learning model provides a portable and publicly accessible mechanism for performing local-ancestry inference. Specifically, while the training data used to train the machine learning model includes datasets containing proprietary human genomic sequences data that are protected by privacy restriction or otherwise not accessible to the public, the trained parameters of the machine learning model (e.g., neural network weights, decision sequences and thresholds of the decision trees, etc.) do not identify individuals and can be made publicly available. As a result, the machine learning model can be made publicly available to perform local-ancestry inference to support various biomedical applications, such as predicting a risk of disease of a subject, determining a link between the subject's genetic makeup with certain biological traits of the subject, determining a treatment for the subject, etc.

Some embodiments are directed to systems and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1A and FIG. 1B illustrates examples of single-nucleotide polymorphism (SNP) in a genome and the ancestral origins of the SNPs;

FIG. 2A, FIG. 2B, FIG. 2C, FIG. 2D, and FIG. 2E illustrate examples of a machine learning model for performing a local-ancestry inference, according to some embodiments;

FIG. 3A, FIG. 3B, and FIG. 3C illustrate example components of the machine learning model of FIG. 2A-FIG. 2C and their operations, according to some embodiments;

FIG. 4A and FIG. 4B illustrate example components of the machine learning model of FIG. 2A — FIG. 2C, according to some embodiments;

FIG. 5A and FIG. 5B illustrate examples of training operations;

FIG. 6A, FIG. 6B, and FIG. 6C illustrate example components of the machine learning model of FIG. 2A-FIG. 2C, according to some embodiments;

FIG. 7A and FIG. 7B illustrate example test results and applications of the machine learning model of FIG. 2A-FIG. 2C, according to some embodiments;

FIG. 8 illustrates an example method of performing a local-ancestry inference, according to some embodiments; and

FIG. 9 illustrates a computer system in which embodiments of this disclosure can be implemented.

### DETAILED DESCRIPTION

Local-ancestry inference uses the pattern of genetic variation observed at various sites along an individual's DNA to estimate the ancestral origin of each segment of an individual's DNA. Because DNA is inherited as an intact sequence with only rare, random swaps in ancestry (between the two parental DNA sequences) at each generation, ancestral SNPs form contiguous segments allowing for powerful ancestry inference based on patterns of contiguous SNP variants.

Embodiments of the present disclosure provide methods, systems, and apparatus for estimating the ancestral origin(s) of segments of genetic variants (e.g., SNPs) in a DNA sequence using a trained machine learning model. The estimation can be at a high resolution, such as at milliMorgan resolution. In one example, a computer-implemented method includes receiving data representing an input genomic sequence of a subject (e.g., a person). The input genomic sequence may cover a plurality of segments each including a plurality of single nucleotide polymorphism (SNP) sites of the genome of the subject. Each segment may be represented in the data by a sequence of SNP values at the SNP sites, with each SNP value specifying a variant at the SNP site. The data can be obtained from a haploid or diploid DNA sequence. The data can be obtained from, for example, a genome sequencing operation that provides a genomic sequence of the subject, a DNA microarray that contains segments of DNAs, etc. The haplotype information in the data can be encoded to include, for example, different values for different variants. A first value can represent that the subject has a common variant (e.g., a value of −1) at a SNP site. A second value can represent that the subject has a minority variant (e.g., a value of +1) at the SNP site. A third value (e.g., a value of 0) can represent that the genomic information is missing at the SNP site. In some examples, a two-bit value can be used to represent common variant (e.g., [0, 1]), minority variant (e.g., [1, 0]), and missing (e.g., [0, 0]).

The method further comprises storing a trained machine learning model, the machine learning model comprising a predictor sub-model and a smoothing sub-model. The machine learning model can be trained based on training genomic sequences and known ancestral origins of the training genomic sequences. Using the predictor sub-model and based on the data, an initial ancestral origin estimate of each segment of SNP values can be determined. Moreover, for each segment of the plurality of segments, a subset of neighboring segments that neighbor the segment in the genome of the subject. The initial ancestral origin estimates for the subset of neighboring segments can be input to the smoothing sub-model to perform a smoothing operation over the segment and the subset of neighboring segments. As a result of the smoothing operation, a final prediction result for the ancestral origin of each segment of the plurality of segments can be determined. The ancestral origin for different parts of the genome of the subject can then be determined based on the final prediction results obtained for each segment. The ancestral origins determined for the different parts of the subject's genome can be provided to various applications to perform other operations, such as predicting the risk of the subject having a certain disease, determining a link between the subject's genetic makeup with certain biological traits of the subject, determining a treatment for the subject, etc.

The predictor sub-model may comprise, for example, one or more classifiers, one or more regressors, etc. A classifier can identify a probability (including binary 0 and 1) that a segment is from a particular ancestral origin; such a probability can be determined for each of a predetermined list of candidate ancestral origin categories. The initial ancestral origin estimate for the segment can be determined as the candidate ancestral origin category having the highest probability. Moreover, a regressor can provide a prediction that maps to geographical coordinates, or other types of identifiers, e.g., for providing accurate results within particular locales that are near each other.

In some examples, each classifier can perform a classification operation on an non-overlapping segment of the SNPs to generate a classifier output. Each classifier can determine a probability of the segment being classified into each candidate ancestral origin categories (e.g., Africa, East Asia, and Europe), and the probabilities output by the classifiers can be combined to output an initial ancestral origin estimate based on the candidate ancestral origin category having the highest probability. In some examples, each regressor can perform a regression operation on a random subset of SNPs of the segment of the SNPs, which can be combined to output one or more origin estimates indicative of an ancestral origin of the segment of the SNPs. The one or more origin estimates can include, for example, the geographical coordinates (e.g., longitude and latitude) of the ancestral origin locale, a code representing the ancestral origin locale, etc. In some cases, a regressor can provide useful ancestral estimates even for closely related populations, which can present problem for a classifier, which treats each ancestry misclassification equally even though some ancestries are much more related than others. The plurality of classifiers and regressors can perform, respectively, the classification operations and regression operations in parallel to support the local-ancestry inference operation in a distributed computing environment, which makes the inference operation more scalable and computationally efficient.

In some examples, the predictor sub-model can include a single prediction model (e.g., a single classifier model, a single regressor model, etc.). The single prediction model can include a single set of model parameters which can be combined with different SNP segments to generate classification outputs or regression outputs (which can include coordinates) for different SNP segments. The single prediction model can also accept, for each SNP segment, a segment index associated with the SNP segment, and combine the model parameters with the SNP index and the SNP segment to perform the prediction. The segment index can indicate a particular set of SNP sites and can be in the form of a single number (e.g., 1, 2, 3, etc.) or in the form of one hot encoding (e.g., [1, 0, . . . 0], [0, 1, . . . 0], etc.). Other types of encoding, such as positional encodings in Transformers neural networks, can also be used to represent the segment index. The segment index allows the single prediction model to perform the prediction differently for different SNP segments using the same set of model parameters.

In addition, the smoothing sub-model can perform a smoothing operation over initial ancestral origin estimates of a subset of neighboring segments. The smoothing operation can remove/reduce discontinuities in the initial ancestral origin estimates between segments introduced by the classifiers or the regressers. The smoothed ancestral origin estimates (classifier outputs, regressor outputs, etc.) of the segments can then be concatenated as determined ancestral origins of different parts of the subject's genome.

Various techniques are proposed to implement the machine learning model. In one example, the trained machine learning model may include one or more neural network models. Specifically, each classifier or regressor of the predictor sub-model may include a fully-connected neural network model. The fully-connected neural network model includes at least an input layer and an output layer. The input layer includes a plurality of input nodes, whereas the output layer includes a plurality of output nodes. Each input node corresponds to a particular SNP site of the segment received by the classifier. Each input node can receive an encoded value (e.g., 1, 0, −1) of a SNP variant at the corresponding SNP site. The input node can scale the corresponding encoded value with a first set of weights to generate a set of scaled encoded values.

Each output node of the output layer can receive inputs based on the scaled encoded values and sum the inputs. Each output node can correspond to one of the plurality of candidate ancestral origins such as, for example, Africa, East Asia, and Europe. Each output node may also apply an activation function to the sum of the inputs to generate an initial ancestral origin estimate. The initial ancestral origin estimate output by an output node can include a value indicative of whether the segment of SNPs processed by the neural network model is classified into the corresponding candidate ancestral origin (e.g., one of Africa, East Asia, and Europe, etc.), such as the probability of the segment of SNPs having the candidate ancestral origin, as described above.

The fully-connected neural network model of the predictor sub-model can be implemented using various neural network architectures. In some examples, the fully-connected neural network model comprises only the input layer and the output layer. Such arrangements allow smaller and less complex classifiers to be implemented in the predictor sub-model, which in turn allows the classifiers to be trained and to perform the classification operations using less time and computation resources. Moreover, the weights of the input layer can specify the importance of each SNPs in identifying the ancestral origin of an SNP segment, which can lead to a more interpretable network.

In some examples, the fully-connected neural network model comprises a hidden layer between the input layer and the output layer. The hidden layer can identify a hidden representation of certain information (e.g., captured in the form of feature vectors) in an input SNP segment, and the hidden representation can be mapped to one of the candidate ancestral origins, or coordinates of an ancestral origin locale. The hidden layer can provide a non-linear mapping between the input SNP segment and an ancestral origin classification output or ancestral origin locale coordinates, which can improve the accuracy of the ancestral origin estimate.

The hidden layer of the fully-connected neural network may comprise a plurality of intermediate nodes. Each intermediate node can receive a scaled encoded value of SNP from each input node, sum the scaled encoded values, and scale the sum with a second set of weights, and apply an activation function on the scaled sums to generate a set of intermediate outputs. The output layer can receive an intermediate output from each intermediate node as an input, and generate an initial ancestral origin estimate based on the intermediate outputs. The intermediate output can include hidden representations that can provide non-linear mapping between the input SNP segment and an ancestral origin classification output or ancestral origin locale coordinates.

In addition, the smoothing sub-model can perform a smoothing operation over subsets of initial ancestral origin estimates from the predictor sub-model to remove/reduce discontinuities in the initial ancestral origin estimates. The smoothing operation can smooth on a per segment basis. For each segment, a subset (e.g., a window) of neighboring segments can be used to determine the ancestral original of a given segment. In some examples, the smoothing sub-model can include a convolutional neural network (CNN) that can perform a convolution operation between a kernel and the initial ancestral origin estimates generated for each segment of the input SNP sequence, and the results of the convolution operation can be output as the final ancestral origin prediction results.

As part of the convolution operation, a kernel comprising an array of weights can be multiplied with the initial ancestral origin estimates of a subset of neighboring segments included in a sliding window. The multiplication results can then be summed to generate a smoothed ancestral origin estimate. The window can center around a target initial ancestral origin estimate to be replaced by the smoothing operation, as well as a pre-determined number of initial ancestral origin estimates in front of and behind the target initial ancestral origin estimate. Each weight included in the kernel can be mapped to an initial ancestral origin estimate. With the convolution operation, the initial ancestral origin estimates can be smoothed by performing a weighted averaging of the initial ancestral origin estimates within a window, which can remove discontinuities in the initial ancestral origin estimates between segments introduced by the predictor sub-model.

In some examples, as part of the weighted averaging operation, the smoothing sub-model can assign weights to each initial ancestral origin estimate based on usefulness metrics of the segment of the SNPs represented by the respective initial ancestral origin estimate. The usefulness metrics can reflect, for example, whether the ancestral origins of the SNP variants at certain SNP sites of the segment can be correctly predicted. The usefulness metrics can be based on, for example, a probability of prediction error of ancestral origin for the segment, which can be determined based on the prior prediction results of the segment of genomes of a population. A smaller weight can be assigned to an initial ancestral origin estimate for a segment of SNPs having a higher probability of prediction error, whereas a larger weight can be assigned to an initial ancestral origin estimate for a segment of SNPs having a lower probability of prediction error. The weights can be part of the kernel of the convolution operation, or can be applied to each initial ancestral origin estimate prior to the convolution operation.

In some examples, the input SNP sequence (e.g., diploid) can include a maternal SNP sequence and a paternal SNP sequence of the subject, and then the CNN can perform the convolution operation between the kernel and the classifier outputs for the maternal and paternal SNP sequences, to generate the final ancestral origin prediction results of ancestral origin(s) for the segments of the maternal and paternal SNP sequences. With such arrangements, the final prediction results can become invariant of the order in which the maternal SNP sequence and the paternal SNP sequence are presented in the input SNP sequence.

In some examples, the predictor sub-model and the smoothing sub-model can include a plurality of decision trees. Specifically, each classifier or regressor of the predictor sub-model can include a first plurality of decision trees. Each decision tree can process a random subset of the sequence of SNPs to generate a decision, and the decisions of the plurality of decision trees can be combined to generate an initial ancestral origin estimate. In a case where the plurality of decision trees form a classifier, each decision tree can output a probability of the segment of SNPs being classified into a particular ancestral origin category based on the random samples, and the probabilities can be averaged to generate the initial ancestral origin estimate. In a case where the plurality of decision trees form a regressor, the decision trees can be trained to generate decision outputs representing a regression model that fits SNPs of training data to the geographical coordinates of a known ancestral origin locale of the SNPs. The decision trees can then process the random subsets of the input sequence of SNPs to output intermediate geographical coordinates. The intermediate geographical coordinates can then be combined (e.g., summed) to generate the initial geographical coordinates estimates of the ancestral origin locale for a subset of the SNPs. The decision trees can perform the regression/classification operations in parallel in a distributed computing environment, which makes the operations more scalable and computationally efficient.

In addition, the smoothing sub-model can also include a second plurality of decision trees to perform the smoothing function. Similar to the convolutional neural network as described above, the smoothing function can be applied on initial ancestral origin estimates of a subset of neighboring segments based on a sliding window approach. The window can center around a target initial ancestral origin that is to be smoothed and can include a pre-determined number of initial ancestral origin estimates before and after the target initial ancestral origin. Different random subsets of the initial ancestral origin estimates within the window can be input to each of the second plurality of decision trees. The decisions output by the decision trees can then be combined to generate a final ancestral origin estimate, which can replace the target initial ancestral origin. The decision trees can be trained to, for example, perform weighted averaging of neighboring initial ancestral origin estimates within a window to remove the discontinuities in the initial ancestral origin estimates. The window can slide/move to cover different subsets of initial ancestral origin estimates to generate final ancestral origin prediction results for different segments of SNPs, similar to the convolution operation performed by a CNN. The weights assigned to each initial ancestral origin estimate can be based on a measurement of usefulness of the segment of the SNPs represented by the respective initial ancestral origin estimate, as explained above.

The machine learning model can be trained to improve the accuracy of prediction. The machine learning model can be trained based on segments of training genomic sequences that have known ancestral origins. Specifically, the machine learning model can be trained based on training data derived from full genome data of a population of known ancestral origins to be identified by the machine learning model. For example, in a case where the machine learning model is to classify a segment of SNPs into one of Africa, East Asia, and Europe, the training data can include genome data of individuals from various locales of Africa, East Asia, and Europe, as well as smaller geographic regions. From the full genomic sequence of these individuals, simulated genomic sequences of simulated admixed descendants of these individuals can be generated based on a simulation (e.g., a Wright-Fisher forward simulation) over a series of generations. A set of training data comprising genomic sequences of simulated admixed descendants of these individuals (e.g., over numerous generations), as well as the known ancestral origins of SNP segments of the simulated genomic sequences, can be used to train and validate the machine learning model. The training allows the machine learning model to learn from the relationships between patterns of SNP variants at different DNA sites and their ancestral origins to perform local-ancestry inference.

The training operation can include a forward propagation operation and a backward propagation operation. As part of the forward propagation operation, the machine learning model can receive training data including sequences of SNPs of known ancestral origins to generate predictions of ancestral origins of the sequences. A comparison between the predicted and true ancestral origin category (or between the predicted and known geographical coordinates of ancestral origin locale) of each SNP segment can be made. Various parameters of the predictor sub-model and the smoothing sub-model, such as the weights of the fully-connected neural network model, the parameters of the kernel of the convolutional neural network model, the decision trees, the weights associated with the SNP segments in the smoothing operations, etc., can be adjusted in the training operation to maximize the matching between the predicted and true ancestral origins.

Various techniques of training the machine learning model are proposed. In a case where the machine learning model operates as a classifier, the training operation can be based on a combined cross-entropy loss function, which can include a linear combination of a first loss function associated with the predictor sub-model and a second loss function associated with the smoothing sub-model. The first loss function can compare the initial ancestral origin estimates output by the predictor sub-model for segments of SNPs in training data with their true ancestral origins to generate first loss gradients, which can be used to adjust the weights or decision thresholds of the predictor sub-model to minimize the first loss function. Moreover, the second loss function can compare the final ancestral origin prediction results output by the smoothing sub-model for the segments of SNPs with their true ancestral origins to generate second loss gradients, which can be used to adjust the kernels or decision thresholds of the smoothing sub-model to minimize the second loss function.

Moreover, in a case where both the predictor sub-model and the smoothing sub-model comprise decision trees to perform a regression operation, the training operation can be based on a gradient tree boosting operation. Specifically, the training operation can start with creating a first decision tree for the first sub-network to fit the first decision outputs (e.g., ancestral origin estimates, geographical coordinates of ancestral origin locales, etc.) with segments of SNPs. A first set of residuals can be determined based on, for example, differences between the predicted ancestral origins from the first decision tree and true ancestral origins, differences between the predicted geographical coordinates of ancestral origin locales from the first decision tree and true geographical coordinates of ancestral origin locales, etc.

A second decision tree can then be generated and trained to fit the second decision outputs over the first set of residuals. For example, the second decision tree can be trained to generate second decision outputs to match the first set of residuals as much as possible, for the same segment of SNPs input to the first decision outputs. A second set of residuals can be determined based on differences between the second decision outputs and the first set of residuals. A third decision tree can then be generated and trained to fit a third decision output over the second set of residuals. The training process can be repeated until, for example, a pre-determined number of trees is reached, a pre-determined threshold level of residuals is achieved, etc. Through the addition of new decision trees to fit the decision tree outputs with the residuals, the decision trees can represent a regression model of a relationship between SNPs and ancestral origin estimates and/or geographical coordinates of ancestral origin locale.

### I. Local-Ancestry Inference Based on SNPS

A single-nucleotide polymorphism (SNP) may refer to a DNA sequence variation occurring when a single nucleotide adenine (A), thymine (T), cytosine (C), or guanine (G) in the genome differs between members of a species.

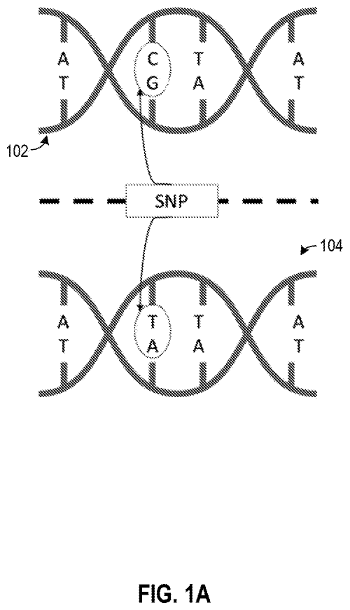

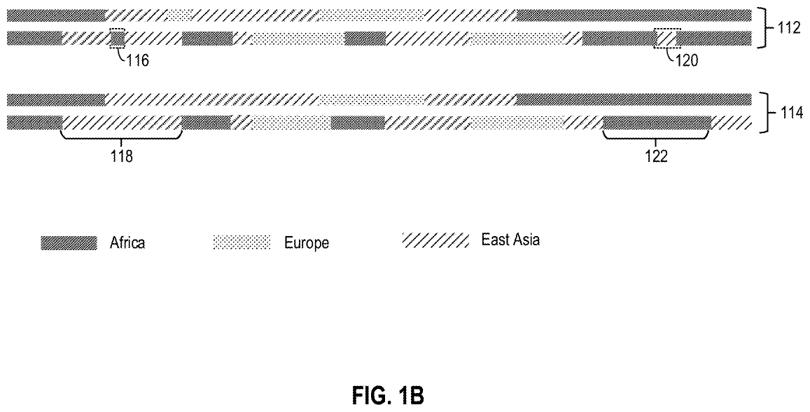

FIG. 1A illustrates an example of SNP. FIG. 1A illustrates two sequenced DNA fragments 102 and 104 from different individuals. Sequenced DNA fragment 102 includes a sequence of base pairs AT-AT-CG-CG-CG-TA-AT, whereas sequenced DNA fragment 104 includes a sequences of base pairs AT-AT-CG-CG-TA-TA-AT. As shown in FIG. 1A, DNA fragments 102 and 104 contain a difference in a single base pair (CG versus TA, typically referred to as C and T) of nucleotides. The difference can be counted as a single SNP. A SNP can be encoded into a value based on whether the SNP is a common variant or a minority variant. The common variant can be more common in the population (e.g., 80%), whereas the minority variants would occur in fewer individuals. In some examples, a common variant can be encoded as a value of −1, whereas a minority variant can be encoded as a value of +1.

Modern human populations, originating from different continents and different subcontinental regions, exhibit discernible differences in the frequencies of SNP variants at each site in the DNA sequence in their genomes, and in the correlations between these variants at different nearby sites, due to genetic drift and differing demographic histories (bottlenecks, expansions and admixture) over the past fifty thousand years. Because DNA is inherited as an intact sequence with only rare, random swaps in ancestry (between the two parental DNA sequences) at each generation, ancestral SNPs form contiguous segments allowing for powerful ancestry inference based on patterns of contiguous SNP variants.

FIG. 1B illustrates an example of distribution of ancestral origins among segments of SNPs of an admixed pair of chromosomes of an individual: one from each parent of the individual. Distribution 112 illustrates the true ancestral origins of genetic material at different SNP sites of the individual. In the example of FIG. 1B, the ancestral origins of the SNPs may include Africa, East Asia, and Europe. Distribution 114 illustrates the decoded ancestral origins of the SNPs, which can be derived from performing a smoothing operation over distribution 112 to remove ancestral origin discontinuities in a segment, such as discontinuity 116 (Africa) in segment 118 (East Asia), discontinuity 120 (East Asia) in segment 122 (Africa), etc.

The ability to accurately infer the ancestry along the genome in high-resolution is important to understand the role of genetics and environment for complex traits, such as predisposition to certain illness, certain biomedical traits (e.g., blood pressure, cholesterol level, etc.). This can be due to populations with a common ancestry sharing complex physical and medical traits. For example, certain ethnic group may have a relatively high mortality of asthma, whereas another ethnic group may have a relatively low mortality of asthma. Elucidating the genetic associations within populations for predisposition to certain illness and biomedical traits can inform the development of treatments, and allow for the building of predictors of disease risk, known as polygenic risk scores. However, because the correlations between neighboring genetic variants (e.g., SNPs) are ancestry dependent, applying these risk scores to an individual's genome requires knowledge of the individual's ancestry at each site along the genome. With the increasing diversity of admixed modern cosmopolitan populations, it becomes increasingly common that an individual's genome has multiple ancestral origins, as shown in the examples of FIG. 1B. As a result, ancestry-specific analysis along the genome is becoming an increasingly complex and important computational problem.

II. Local-Ancestry Inference using Machine Learning Model

A machine learning model can be used to provide an accurate and publicly accessible mechanism to perform ancestry-specific analysis of a subject's genome data. Specifically, a machine learning model can be trained using genome data of individuals with known ancestral origins to learn various ancestry-specific patterns of SNPs, and to apply the learning to identify ancestry-specific patterns of SNPs from input genome data in more accurate manner. Moreover, while the training data used to train the machine learning model includes datasets containing proprietary human genomic sequences data which is protected by privacy restrictions or otherwise not accessible to the public, the trained parameters of the machine learning model do not identify individuals and can be made publicly available. Therefore, the machine learning model can be made publicly available to perform local-ancestry inference to support various biomedical applications, such as predicting a risk of disease of a subject, determining a link between the subject's genetic makeup with certain biological traits of the subject, determining a treatment for the subject, etc.

A. General Topology

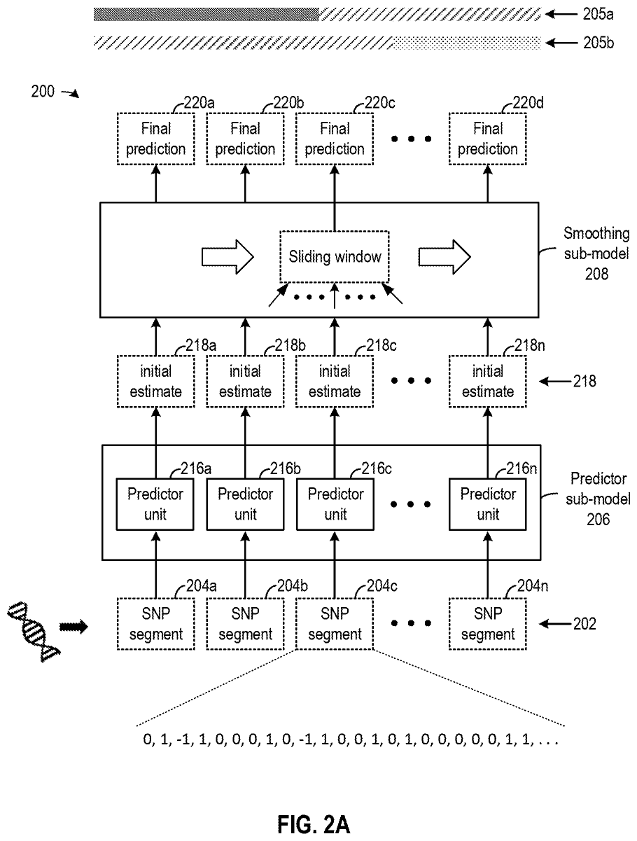

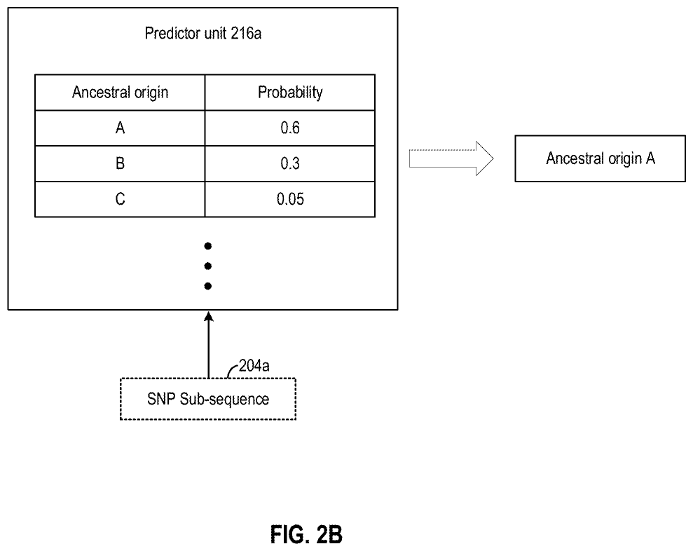

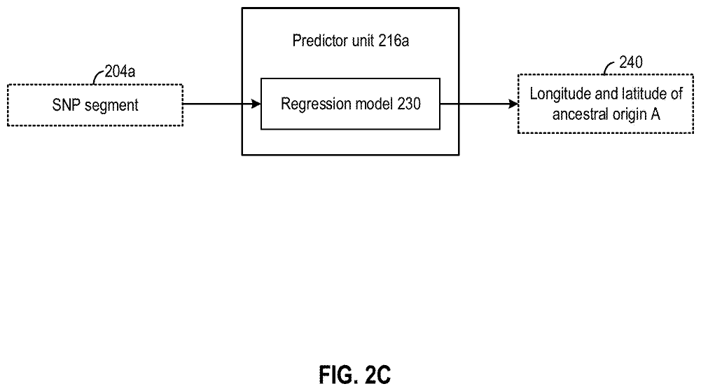

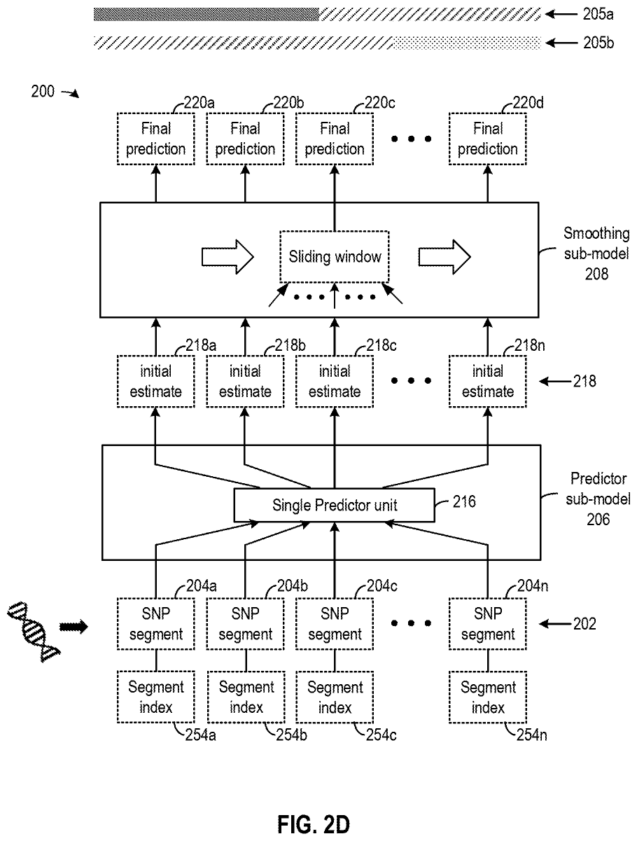

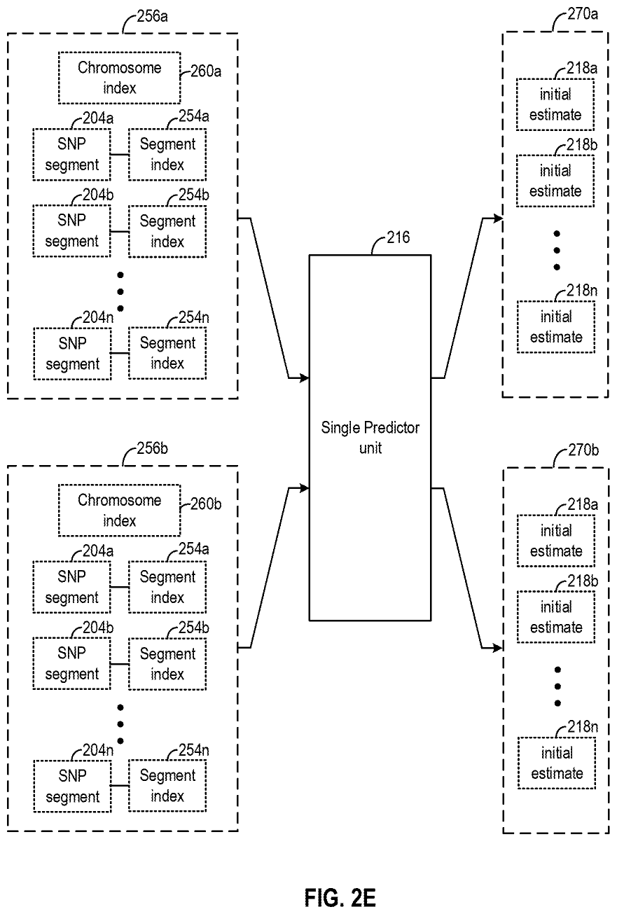

FIG. 2A illustrates a general topology of a machine learning model 200 for performing a local-ancestry inference, according to some embodiments. As shown in FIG. 2A, machine learning model 200 can receive data 202 representing an input genomic sequence of a subject (e.g., a person). The input genomic sequence may cover a plurality of segments each including a plurality of single nucleotide polymorphism (SNP) sites of the genome of the subject. Each segment may be represented, in data 202, by a sequence of SNP values at the SNP sites, with each SNP value specifying a variant at the SNP site. The data can be obtained from a haploid or a diploid DNA sequence. Data 202 can be obtained from, for example, a genome sequencing operation that provides a genomic sequence of the subject, a DNA microarray which contains segments of DNAs, etc. The haplotype information can be encoded to include, for example, a first value representing that a particular SNP is a common variant (e.g., a value of −1) at an SNP site, a second value representing that the SNP is a minority variant (e.g., a value of +1) at the SNP site, or a third value (e.g., a value of 0) representing that the genomic information is missing at the SNP site. Data 202 can be divided into non-overlapping segments, including segments of SNPs 204a, 204b, 204c, 204n, etc. In some examples, each segment can include 500 SNPs. Machine learning model 200 can process data 202 including a maternal haploid DNA sequence and a paternal haploid DNA sequence separately, and generate ancestral origin predictions 205a and 205b for segments of SNPs of each sequence.

In some examples, machine learning model 200 may include two sub-models, including a predictor sub-model 206 and a smoothing sub-model 208. Predictor sub-model 206 can include a plurality of predictor units, including predictor units 216a, 216b, 216c, . . . 210n. Each predictor unit 216 can have a set of model parameters which can be combined with SNP values within a segment of SNPs 204 to generate an initial ancestral origin estimate 218 for the segment of SNPs. For example, predictor unit 216a can generate an initial ancestral origin estimate 218a for a segment of SNPs 204a, predictor unit 216b can generate an initial ancestral origin estimate 218b for a segment of SNPs 204b, predictor unit 216c can generate an initial ancestral origin estimate 218c for a segment of SNPs 204c, whereas predictor unit 216n can generate an initial ancestral origin estimate 218n for a segment of SNPs 204n. As described below, initial ancestral origin estimate 218 can include different types of information, such as a probability of having certain ancestral origin, geographical coordinates of an ancestral origin locale, coordinates in a multi-dimensional space representing ancestry and genetic information, a feature vector containing an ancestry representation, etc.

Each predictor unit 216 can have different model parameters specific for a particular set of SNP sites corresponding to the SNP segments. For example, predictor unit 216a can have a set of model parameters specific for the SNP sites corresponding to segment of SNPs 204a, whereas predictor unit 216b can have a different set of model parameters specific for the SNP sites corresponding to segment of SNPs 204b. As to be described below, the model parameters of each predictor unit 216 can be trained based on training data which include segments of SNPs of known ancestral origins at the corresponding SNP sites. The predictor units can operate in parallel, which allow the operations of the predictor units to be performed in a distributed computing environment, which makes the operations of the predictor units more scalable and computationally efficient. In some examples, different predictor sub-models 206, each having a different set of model parameters for predictor units 216a-216n, can be used to process segments of SNPs from different chromosomes.

In addition, smoothing sub-model 208 can perform a smoothing operation over initial ancestral origin estimates 218 corresponding to multiple neighboring segments to generate final prediction results 220, such as final prediction results 220a, 220b, 220c, 220n, etc. Final prediction results 220 can also include a prediction of a probability of having certain ancestral origin, geographical coordinates of an ancestral origin locale, generalized coordinates in a multi-dimensional space representing ancestry/breed and genetic information, etc. Each final prediction result can be generated for a segment of SNPs, and the final prediction results can be concatenated to become final prediction results of ancestral origins of different parts of the subject's genome, including ancestral origin predictions 205a and 205b. The smoothing operation can remove/reduce discontinuities in the initial ancestral origin estimates between segments. In some examples, smoothing sub-model 208 can also receive feature vectors containing ancestry representations, and then generate final prediction results 220 based on the feature vectors. In some examples, smoothing sub-model 208 can also generate final prediction results 220 based on feature vectors as well as initial ancestral origin estimate 218 of probabilities, geographical coordinates, generalized coordinates, etc.

As shown in FIG. 2A, the smoothing operation can include performing a weighted sum/average of subsets of the initial ancestral origin estimates 218 in a sliding window to generate a final prediction result, and the final prediction results can be output instead of the initial ancestral origin estimates. The sliding window can center around a target initial ancestral origin estimate to be replaced by the final prediction result. For example, to generate final prediction result 220c, which is to replace initial ancestral origin estimate 218c, the sliding window can include initial ancestral origin estimate 218c, as well as a pre-determined number of initial ancestral origin estimates 218 in front of and behind initial ancestral origin estimate 218c.

A. Local-Ancestry Interference based on Classifier and Regression

Predictor sub-model 206 can employ various techniques to generate initial ancestral origin estimates for segments of SNPs, such as performing classification and regression operations. When operating as a classifier, the predictor unit can use the encoded SNP values at the SNP sites in a SNP segment to compute a probability of the SNP segment having an ancestral origin. The ancestral original can be selected from a set of candidate ancestral origins. The predictor unit can then classify the SNP segment as having the ancestral origin associated with the highest probability.

FIG. 2B illustrates an example classification operation. As shown in FIG. 2B, predictor unit 216a can compute the probabilities of SNP segment 204a having ancestral origins A, B, C, etc.

Predictor unit 216a can generate outputs in various forms. In one example, predictor unit 216a can generate a classification output that classifies segment 204a into the ancestral origin having the highest probability. In another example, predictor unit 216a can generate the classification output in one-hot encoding format, with a logical one assigned to the ancestral origin having the highest probability and a logical zero assigned to the rest of ancestral origin. In FIG. 2B, ancestral origin A has the highest probability, therefore, predictor unit 216a can output ancestral origin A or [1, 0, 0] (with 1 representing ancestral origin A) for segment 204a. In some examples, predictor unit 216a can also output the probabilities directly to the smoothing layer or other types of numerical outputs including a logit value for each probability, a score of belonging to a class (as in Support Vector Machines), etc. In some examples, predictor unit 216a can also generate a feature vector containing an ancestry representation. For example, the feature vector can include an array of probability values, with each probability value for an ancestral origin.

B. Local-Ancestry Interference based on Regression

In a case where a predictor unit operates as a regressor, the predictor unit can store a regression model that relates various patterns of SNP values at pre-determined SNP sites to geographical coordinates of ancestral origin locales. The regression model can include a model parameter mapped to each SNP site. The model parameters can be combined with a sequence of SNP encoded values at the SNP sites to compute geographical coordinates of an ancestral origin locale of an SNP segment. The regression model can be trained based on, for example, minimizing a distance between the predicted geographical coordinates of the ancestral origin locale of a SNP segment and the known geographical coordinates for a population of subjects.

FIG. 2C illustrates an example regression operation. As shown in FIG. 2C, predictor unit 216a can input SNP segment 204a into regression model 230 to compute the geographical coordinates 240 (e.g., longitude and latitude), or other identifying information, of the ancestral origin locale of SNP segment 204a. Geographical coordinates 240 can indicate a location within, for example, ancestral origin A.

In some examples, as shown in FIG. 2D, instead of having a plurality of predictor units 216a-n each having a different model parameter, predictor sub-model 206 can include a single predictor unit 216 including a single set of model parameters (e.g., a single classifier, a single regressor, etc.) to generate an initial ancestral origin estimate 218 for different SNP segments. Compared with FIG. 2A where different predictor units having different model parameters are used to process the SNP segments, the arrangements in FIG. 2D can reduce the total size of model parameters, which allows predictor sub-model 206 as well as machine learning model 200 to be more compact and requires less memory resources.

As shown in FIG. 2D, single predictor unit 216 can accept, in addition to SNP values of a SNP segment, a segment index 254 associated with the SNP segment as inputs. Each segment index can indicate the SNP sites of a particular SNP segment. The segment index can be combined with the single set of model parameters to generate an initial ancestral origin estimate 218, which allows single predictor unit 216 to perform ancestral origin predictions differently for different sets of SNP sites using the same set of model parameters. For example, single predictor unit 216 can generate initial origin estimate 218a based on SNP segment 204a and a segment index 254a. Moreover, initial origin estimate 218b can be generated based on SNP segment 254b and a segment index 254b. Moreover, initial origin estimate 218c can be generated based on SNP segment 204c and a segment index 254c. As to be described below, the different segment indices can be part of the training data to train single predictor unit 216 to perform ancestral origin predictions differently for different sets of SNP sites.

In some examples, a single predictor unit 216 having a single set of model parameters can process SNP values of SNP segments of different chromosomes to generate initial estimates 218 for the different chromosomes. In addition to segment indices, single predictor 216 can also accept a chromosome index associated with a particular chromosome. The chromosome index allows single predictor 216 to generate initial estimates of ancestral origins differently for different chromosomes using the same set of model parameters. For example, as shown in FIG. 2E, single predictor unit 216 can accept inputs 256a for a first chromosome and generate a set of initial estimates 270a for the first chromosome. Moreover, single predictor unit 216 can accept inputs 256b for a second chromosome and generate a set of initial estimates 270b for the second chromosome. Inputs 256a can include SNP segments 204a-204n each associated, respectively, segment indices 254a-254n. In addition, inputs 256a also include a chromosome index 260a associated with the first chromosome. In addition, inputs 256b can include SNP segments 204a-204n each associated, respectively, segment indices 254a-254n. In addition, inputs 256b can also include a chromosome index 260b associated with the second chromosome. The arrangements of FIG. 2E allow one set of model parameters to be reused not only between different SNP segments but also between different chromosomes, which allows predictor sub-model 206 as well as machine learning model 200 to be even more compact and requires less memory resources.

Machine learning model 200 can be implemented using various techniques. In some examples, each classifier or regressor of predictor sub-model 206 may include a fully-connected neural network model, which may include a hidden layer, while smoothing sub-model 208 may include a convolutional neural network (CNN). In some examples, each classifier or regressor of predictor sub-model 206, as well as smoothing sub-model 208, may include a plurality of decision trees.

C. Fully-Connected Neural Network As Predictor Sub-Model

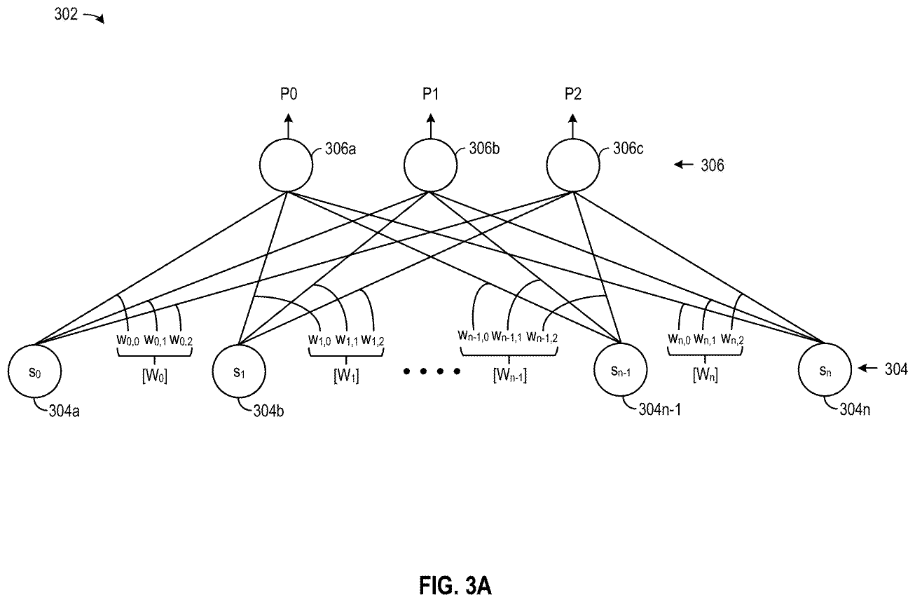

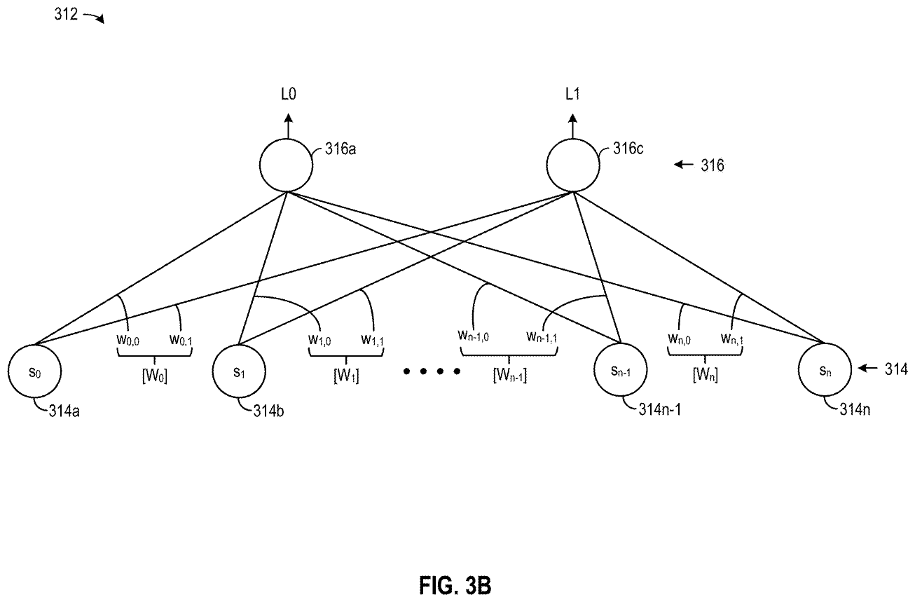

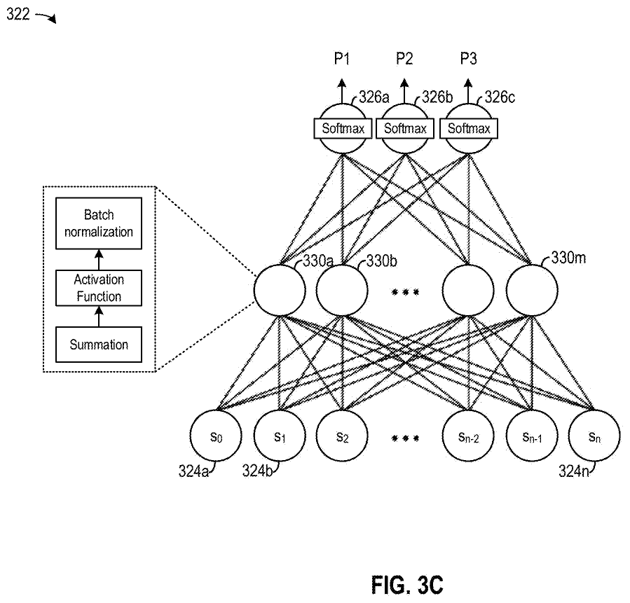

FIG. 3A-FIG. 3C illustrate examples of predictor sub-model 206 implemented using an artificial neural network model. Artificial neural networks are computing systems with an architecture based on biological neural networks. An artificial neural network can include a set of weights. Through computations, the weights can be combined with input data to extract information, and an output (e.g., a decision, a computed value, etc.) can be made based on the extracted information. Examples of neural networks can include a fully-connected neural network, a convolutional neural network, a recurrent neural network (e.g., a Long Short Term Memory (LSTM) network, a Gated Recurrent Unit (GRU) network, self-attention layers, transformer layers, residual blocks, etc. Predictor sub-model 206 can be implemented using any of these neural networks. In FIG. 3A-FIG. 3C, an example of predictor sub-model 206 implemented using a multi-layer fully-connected neural network is illustrated.

1. Two-Layer Neural Network

FIG. 3A illustrates an example of predictor 216a including a neural network 302 and trained as a classifier. Neural network 302 includes an input layer 304 and an output layer 306. Input layer 304 includes a plurality of input nodes such as, for example, input nodes 304a, 304b, . . . 304n. Moreover, output layer 306 includes a plurality of output nodes such as, for example, output nodes 306a, 306b, and 306c.

Each input node receives an encoded value (e.g., 1, 1, −1) of an SNP value at a particular SNP site of the segment received by the classifier. For example, input node 304a receives an encoded value so, input node 304b receives an encoded value Si, whereas input node 304n receives an encoded value sₙ. Each input node is associated with a set of weights. For example, input node 304a is associated with a set of weights W₀, input node 304b is associated with a set of weights W₁, whereas input node 304n is associated with a set of weights Wₙ. Each input node can scale the input encoded value with the associated set of weights to generate a set of scaled encoded values, and transmit the scaled encoded values to output nodes of output layer 306. In a case where predictor 216a receives a segment of 500 SNPs, input layer 304 can include 500 input nodes.

In FIG. 3A, neural network 302 can be a fully-connected neural network, and each output node of output layer 306 is connected to each input node of input layer 304 and receives scaled encoded values from each input node. Specifically, each set of weights of an input node can include a weight element corresponding to each output node to generate a scaled encoded value for each output node. For example, set of weights W₀ of input node 304a includes weight elements w_{0,0}, w_{0,1}, and w_{0,2} which correspond to, respectively, output nodes 306a, 306b, and 306c. Moreover, set of weights W₁ of input node 304b includes weight elements w_{1,0}, w_{1,1}, and W_{1,2} which also correspond to, respectively, output nodes 306a, 306b, and 306c. Further, set of weights W₁ of input node 304n includes weight elements w_{n,0}, w_{n,1}, and w_{n,2} which also correspond to, respectively, output nodes 306a, 306b, and 306c.

Each output node can correspond to a candidate ancestral origin category. Each output node can compute a probability of an input SNP segment, represented by a sequence of encoded values s₀, s₁, sₙ, etc., being classified into a candidate ancestral origin category corresponding to the output node. For example, in a case where a set of candidate ancestral origins includes Africa, Europe, and East Asia, output node 306a can output a probability of an input SNP segment being classified into the African origin, output node 306b can output a probability of the input SNP segment being classified into the European origin, whereas output node 306c can output a probability of the input SNP segment being classified into the East Asian origin.

Each output node can receive the scaled encoded values from each input node and sum the scaled values to generate an intermediate sum, which can then be used to compute a probability of the input SNP sequence having the candidate ancestral origin corresponding to the output node. For example, output node 306a can compute an intermediate sum, sum₃₀₆ₐ , as follows:

Σ₃₀₆ₐ=Σᵢ₌₀ⁿ(w_{0,i}×sᵢ) (Equation 1)

In Equation 1, sᵢ represents the encoded SNP value received by each input node (e.g., s₀, s₁, etc.), whereas w_{0,i} represents the weight element in the weight set of each input node corresponding to output node 306a, including weight element w_{0,0} of weights set W₀, weight element w_{1,0} of weights set W₁, etc.

Each output node also implements an activation function which defines the output of that node given the intermediate sum. The activation function can mimic the decision making of a biological neural network. One example of activation function implemented by output nodes 306 may include a Sigmoid function defined according to the following equation:

$$Sigmoid ( x ) = \frac{1}{1 + {e}^{- x}} \qquad ( Equation 2 )$$

In addition to sigmoid, other forms of activation function can also be used included, for example, a ReLU function, a softmax function, a softplus function (which can be a smooth approximation of a ReLU function), a hyperbolic tangent function (tanh), an arc tangent function (arctan), a sigmoid function, a Gaussian function, etc. Example Equations for ReLU and softmax functions are provided below:

$$R e L U ( x ) = \{ \begin{matrix}x & for x \geq 0 \\ 0 & for x < 0\end{matrix} \qquad ( Equation 3 )$$

$$Softmax ( {x}_{i} ) = \frac{{e}^{{x}_{i}}}{{\sum }_{j = 1}^{K} {e}^{{x}_{j}}} \qquad ( Equation 4 )$$

In Equation 4, the input to the softmax function, xᵢ, is an element of a vector having K elements (x₁, x₂, . . . x_{K}).

Each output node can then apply an activation function, such as a sigmoid function, a softmax function, etc., to the intermediate sum to compute a probability of the input SNP sequence having the candidate ancestral origin corresponding to the output node. Other activation function can also be used to compute a feature vector from a set of intermediate sums. For example, output node 306a can compute the probability P0 of the input SNP sequence having the African origin as follows:

P0=Sigmoid(Σ₃₀₆a) (Equation 5)

Output nodes 306b and 306c can also compute, respectively, probability P1 of the input SNP sequence having the European origin, as well as the probability of P2 of the input SNP sequence having the East Asian origin, based on Equation 5.

FIG. 3B illustrates an example of predictor 216a including a neural network 312 and trained as a regressor. Neural network 312 includes an input layer 314 and an output layer 316. Input layer 314 includes a plurality of input nodes such as input nodes 314a, 314b, . . . 314n, each mapped to an encoded value of an SNP sequence, as in FIG. 3A. Moreover, output layer 316 includes a plurality of output nodes 316a and 316b. Each output node can correspond to a component of geographical coordinates of an ancestral origin locale. For example, output node 316a can output the longitude L0, whereas output node 316b can output the latitude L1. Each input node is associated with a set of weights, each including two weight elements corresponding to, respectively, output nodes 316a and 316b. Each input node can scale the input encoded value with the associated set of weights to generate two encoded values for output nodes 316a and 316b. Each output node can sum the scaled encoded values received from input nodes 314a . . . 314n, as in Equation 1, to generate the corresponding component of geographical coordinates of the ancestral origin locale.

In some examples, neural network 312 can also be trained to generate coordinates, or codes, to represent an ancestral origin/breed. As to be described below, the coordinates can be defined in a multi-dimensional space that is defined by dimensions obtained from a dimensionality reduction operation. Neural network 312 can be trained using vectors representing full genomic sequence of pure breed subjects, or subjects having a single ancestral origin for all SNP segments, and reference coordinates in the multi-dimensional space obtained via a dimensionality reduction operation on the vectors. In such examples, output nodes 316a and 316b can output coordinates representing an ancestral origin, or a breed, of a particular SNP segment, and the coordinates may or may not represent a geographical locale but a breed locale, or breed coordinates. The breed coordinates of a particular breed (e.g., of crops or an animal) can be generated from genomic sequences of a pure bred (i.e., known ancestral origin). For example, SNP sites can be encoded (e.g., 0 or 1), and a dimension reduction can be performed, e.g., using principal component analysis (PCA). These breed coordinates can be used as the output label for supervised training, e.g., in a similar manner as geographical coordinates can be used, but in a more generalized sense.

2. Hidden Layer

In some examples, the fully-connected neural network model of predictor sub-model 206 may include a hidden layer between the input layer and the output layer. The hidden layer can classify the input segment of SNPs into a candidate locale of a candidate ancestral origin. The output layer can then further classify the input segment of SNPs into the candidate ancestral origin based on the locales output by the hidden layer. The hidden layer can also provide additional regression parameters for computation of the geographical coordinates of the ancestral origin locale.

FIG. 3C illustrates an example of predictor 216a including a neural network 322 having an input layer 324 and an output layer 326, as well as a hidden layer 328 between input layer 324 and output layer 326. Input layer 324 includes a plurality of input nodes including input nodes 324a, 324b, . . . 324n. Each input node of input layer 324 receives an encoded value (e.g., 1, 1, −1) of an SNP value at a particular SNP site of the segment received by the classifier. For example, input node 324a receives an encoded value so, input node 324b receives an encoded value S₁, whereas input node 324n receives an encoded value sₙ. In a case where input layer 324 receives 500 SNPs in a segment, input layer 324 may include 500 input nodes. Moreover, output layer 326 includes a plurality of output nodes, including output nodes 326a, 326b, and 326c. In FIG. 3C, neural network 322 can be configured as a classifier, and each output node can correspond to a candidate ancestral origin as in neural network 302. In a case where neural network 322 is configured as a regressor, each output node can correspond to a component of geographical coordinates of an ancestral origin locale. Output layer 326 may include two output nodes as in neural network 312.

In addition, hidden layer 328 includes a plurality of intermediate nodes including, for example, intermediate nodes 330a, 330b, 330m, etc. Each intermediate node can receive a scaled encoded value of SNP from each input node, sum the scaled encoded values, and scale the sum with a second set of weights, and apply an activation function on the scaled sums to generate a set of intermediate outputs. The output layer can receive an intermediate output from each intermediate node as an input, and generate an initial ancestral origin estimate (e.g., a classification output, coordinates of an ancestral origin locale, etc.) based on the intermediate outputs. The intermediate output can include hidden representations/features to provide non-linear mapping between the input SNP segment and an ancestral origin classification output (in a case where neural network 322 is configured as a classifier) or ancestral origin locale coordinates (in a case where neural network 322 is configured as a regressor). In some examples, hidden layer 328 includes 30 intermediate nodes.

In the example of FIG. 3C, neural network 322 can be a fully-connected neural network, in which each intermediate node of hidden layer 328 receives input from, and is connected with, each input node of input layer 324, and each output node of output layer 326 receives input from, and is connected with, each intermediate node of hidden layer 328. Specifically, each input node of input layer 324 is associated with a set of weights each corresponding to an intermediate node of hidden layer 328 to generate a set of encoded SNP values, and each input node transmits a scaled encoded SNP value to one of the intermediate nodes of hidden layer 328. Each intermediate node can sum the scaled encoded SNP values to generate a sum (e.g., based on Equation 1) and apply an activation function, such as a sigmoid function, a softmax function, a ReLU function, etc., to generate an intermediate output. Moreover, an optional batch normalization process can be performed at each node to normalize the intermediate outputs to, for example, increase the speed, performance, and stability of neural network 322. The normalization process can include, for example, subtracting a mean of the intermediate outputs from each intermediate output, and dividing by the subtraction results by the standard deviation of the intermediate outputs, to generate a normalized intermediate output. In some examples, the normalization operation can be performed prior to applying the activation function.

Each output node of output layer 326 can receive a normalized intermediate output from each intermediate node of hidden layer 328. The normalized intermediate output received by each output node can represent, for example, a distribution of probabilities of the input SNP sequence having the ancestral origin in each locale represented by each intermediate node, a regression parameter associated with a locale, etc. In a case where neural network 322 is configured as a classifier, each output node can perform a softmax function on the normalized intermediate output. Based on the distribution of probabilities, the softmax function can map hidden layer 328 to the probabilities for assignment to each of the candidate ancestral origins represented by output nodes 326a, 326b, and 326c (e.g., Africa, Europe, and East Asia). As in neural network 302 of FIG. 3A, neural network 322 can output an ancestral origin having the highest probability for the input SNP sequence, the probabilities for each candidate ancestral origin, etc. Each output node can also perform other activation function, such as ReLU, to generate a feature vector.

Neural network 322 can also be trained as a regressor, or to generate coordinates defined in a multi-dimensional space obtained from a dimensionality reduction operation. In both cases, each intermediate node of hidden layer 328 can provide a non-linear mapping between the input SNP sequence to intermediate outputs representing ancestral origin locale coordinates, or coordinates in the multi-dimensional space obtained from a dimensionality reduction operation.

D. Convolutional Neural Network As Smoothing Sub-Model

As described above, in addition to predictor sub-model 206, machine learning model 200 further includes smoothing sub-model 208 to perform a smoothing operation over subsets of initial ancestral origin estimates (e.g., a classification of an ancestral origin, geographical coordinates of an ancestral origin, etc.) generated by predictor sub-model 206, to remove/reduce discontinuities in the initial ancestral origin estimates. In some examples, smoothing sub-model 208 can include a convolutional neural network (CNN) which can perform a convolution operation between a kernel and the initial ancestral origin estimates generated for each segment of the input SNP sequence, and the results of the convolution operation can be output as the final ancestral origin prediction results. Other neural network topologies, such as recurrent neural networks (e.g., LSTM and GRU), self-attention layers, transformer layers, residual blocks, etc., can also be used to implement smoothing sub-model 208.

1. Smoothing Operation

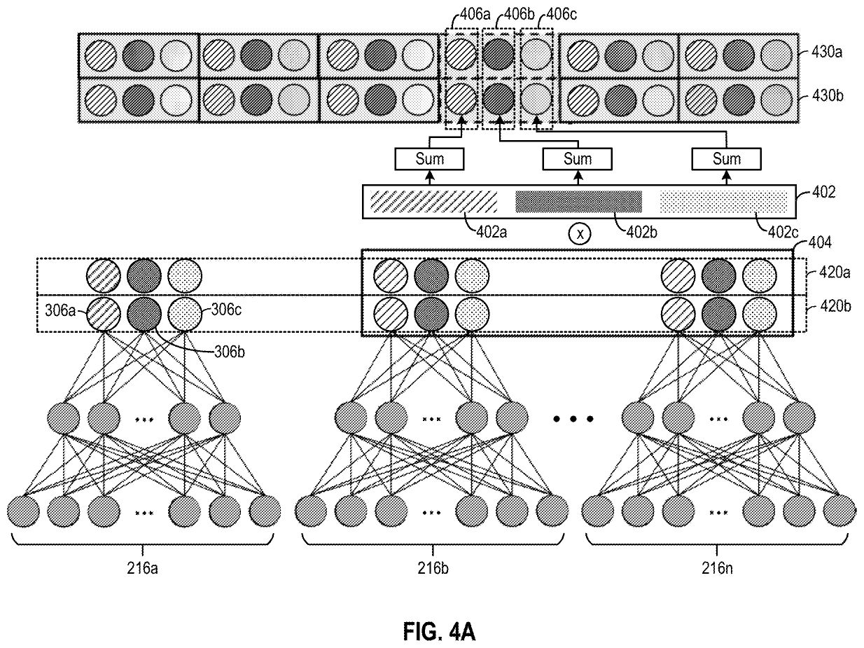

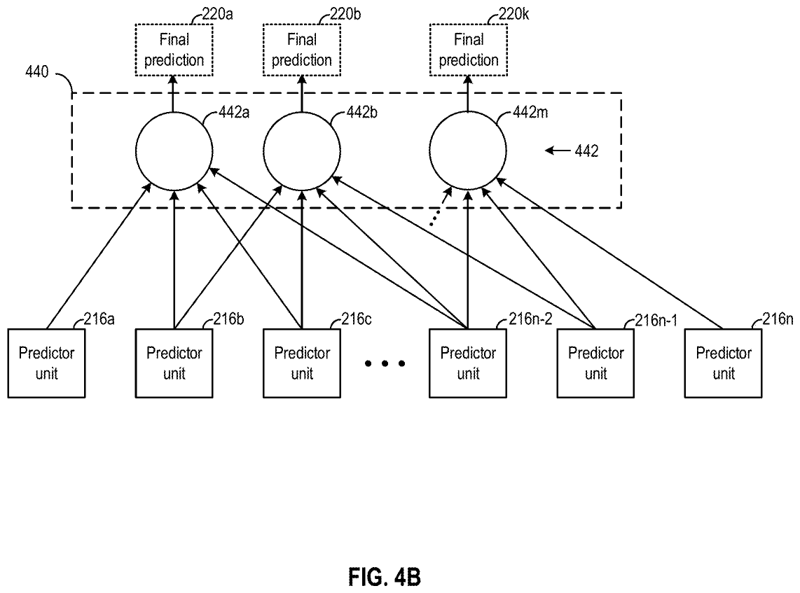

FIG. 4A illustrates an example smoothing operation to be performed by a smoothing sub-model 208. A kernel 402 can operate on initial ancestral origin estimates and/or feature vectors generated from a subset of neighboring SNP segments, with the initial ancestral origin estimates included in a sliding window 404. Specifically, kernel 402 may include an array of weights each corresponding to an initial ancestral origin estimate in sliding window 404. The weights can be multiplied with the corresponding initial ancestral origin estimates, and the products can be summed to generate a final ancestral origin prediction result (e.g., final ancestral origin prediction result 406) for an SNP segment. The final ancestral origin prediction result can replace a target initial ancestral origin estimate of the segment as the output of machine learning model 200. Sliding window 404 can include the target initial ancestral origin estimate to be replaced by the smoothing operation, as well as a pre-determined number of initial ancestral origin estimates in front of and behind the target initial ancestral origin estimate. In some examples, sliding window 404 can include 75 neighboring initial ancestral origin estimates, and the window's position changes for different target initial ancestral origin estimates.

Kernel 402 may include multiple sub-kernels, with each sub-kernel representing a channel and including an array of weights. Each channel can correspond to an output node of output layer 306. For example, kernel 402 may include a sub-kernel 402a, a sub-kernel 402b, and a sub-kernel 402c. Each sub-kernel can operate on the initial ancestral origin estimates from an output node within sliding window 404. Each weight of a sub-kernel can be multiplied with a corresponding initial ancestral origin estimate to generate a product, and the products can be summed to generate a final ancestral origin prediction result. The final prediction result can represent a weighted average of the initial ancestral origin estimates in the window. For example, sub-kernel 402a can be used to generate a weighted average of initial ancestral origin estimates output by output node 306a within sliding window 404. Moreover, a sub-kernel 402b can be used to generate a weighted average of initial ancestral origin estimates output by output node 306b within sliding window 404. Further, a sub-kernel 402c can also be used to generate a weighted average of initial ancestral origin estimates output by output node 306c within sliding window 404.

In some examples, as part of the weighted averaging operation, smoothing sub-model 208 can assign weights to each initial ancestral origin estimate based on usefulness metrics of the segment of the SNPs represented by the respective initial ancestral origin estimate. The usefulness metrics can reflect, for example, whether the ancestral origins of the SNP variants at certain SNP sites of the segment can be correctly predicted. The usefulness metrics can be based on, for example, a probability of prediction error of ancestral origin for the segment, which can be determined based on the prior prediction results of the segment of genomes of a population. A smaller weight can be assigned to an initial ancestral origin estimate for a segment of SNPs having a higher probability of prediction error, whereas a larger weight can be assigned to an initial ancestral origin estimate for a segment of SNPs having a lower probability of prediction error. The weights can be part of kernel 402, or can be applied to each initial ancestral origin estimate prior to being multiplied with kernel 402.

In some examples, input SNP sequence, such as data 202 of FIG. 2A, can include a maternal SNP sequence and a paternal SNP sequence of the subject. The maternal and paternal SNP sequences can be separately processed by predictor sub-model 206 to generate initial ancestral origin estimates 420a and 420b for the maternal SNP sequence and the paternal SNP sequence. Each of sub-kernels 402a, 402b, and 402c can include weights, with each weight corresponding to an initial ancestral origin estimate output by an output node for both the maternal SNP sequence and the paternal SNP sequence. The weights can be multiplied with the corresponding initial ancestral origin estimates to generate two sets of sums, and a final prediction result for each of the maternal SNP sequence and the paternal SNP sequence can be generated. As a result, two sets of final prediction results, including final prediction results 430a and 430b, can be generated for the maternal SNP sequence and the paternal SNP sequence. Final prediction results 430a and 430b for the segments can then be concatenated to become ancestral origin predictions 205a and 205b for segments of SNPs of each sequence. With such arrangements, the final prediction results can become invariant of the order in which the maternal SNP sequence and the paternal SNP sequence are presented in the input SNP sequence.

2. Convolutional Neural Network

FIG. 4B illustrates an example of a convolutional neural network (CNN) 440 that can be part of smoothing sub-model 208. CNN 440 can include a layer 442 including nodes 442a, 442b, 442m, etc. Each node of layer 442 can be connected to a subset of predictor units 216 of predictor sub-model 206 according to sliding window 404. For example, in a case where sliding window 404 includes 75 initial ancestral origin estimates, node 442a can be connected to 75 predictor units starting from predictor unit 216a, node 442b can be connected to 75 predictor units starting from predictor unit 216b, whereas node 442m can be connected to 75 predictor units ending at predictor unit 216n. Each node of layer 442 can implement kernel 402 and generate a final prediction result, including final prediction results 220a, 220b, 220k, etc., for an output node. Specifically, each node of layer 442 can generate a weighted average of the initial ancestral origin estimates output by overlapping groups of predictor units which represent the sliding window. For example, node 442a receives inputs from predictor units 216a, 216b, 216c, . . . 216n-2, whereas node 442b receives inputs from predictor units 216b, 216c, . . . 216n-1. In some examples, the convolution operation can be performed with proper reflection padding to maintain the same input and output size. For example, in a case where there are fewer than 75 initial ancestral origin estimates available (e.g., at the beginning or end of a chromosome) to be input to a node of layer 442, reflection padding can be applied (e.g., by zero padding) to replace missing initial ancestral origin estimates as inputs to the node. In a case where CNN 440 receives n initial ancestral origin estimates from predictor units 216, CNN 440 can also generate n final prediction results based on reflection padding.

E. Training of Neural Network Sub-Models

Machine learning 200 can be trained to improve the accuracy of predictions. Machine learning model 200 can be trained based on training data derived from full genome data of population of known ancestral origins to be identified by the machine learning model. For example, in a case where the machine learning model is to classify a segment of SNPs into one of East Asia, Africa, and Europe, the training data can include genome data of individuals from various locales of East Asia, Africa, and Europe such as, for example, China, Japan, South Korea, England, France, Spain, South Africa, Egypt, etc.

From the full genomic sequence of these individuals, simulated genomic sequence of simulated admixed descendants of these individuals is generated based on Wright-Fisher forward simulation over a series of generations, such as after 2, 4, 16, 32, and 64 generations. With increasing numbers of generations following initial admixture, the simulated descendants have increasing numbers of ancestry switches along the genome, which can lead to a more challenging inference operation. A set of training data comprising genomic sequence of simulated admixed descendants of these individuals with a wide range of generations, as well as the known ancestral origins of SNP segments of the simulated genomic sequences, can be used to train and validate the machine learning model, which allows the machine learning model to learn from the relationships between patterns of SNP variants at different SNP sites and their ancestral origins reflected in the training data to perform local-ancestry inference. To improve the robustness of the trained machine learning model in handling missing SNP data, dropout regularization can be applied to the training data to model missing input SNPs, which is a common occurrence if the input data is from genotyping arrays, such as DNA microarrays.

In addition, in a case where the predictor sub-model includes a plurality of predictor units each to process an SNP segment at a corresponding set of SNP sites, each predictor unit can be trained based on SNP data at the corresponding set of SNP sites, and each predictor unit can include a different set of model parameters (e.g., weights, decision tree topology, decision criteria, etc.) as a result of the training. In a case where the predictor sub-model include a single predictor unit, the predictor sub-model can be trained based on SNP segment data as well as their associated segment indices to enable to sub-model to distinguish different sets of SNP sites as part of the learning. This allows the predictor sub-model to perform predictions differently for different SNP sites using the same set of model parameters.

The training operation can include a forward propagation operation and a backward propagation operation. As part of the forward propagation operation, the machine learning model can receive training data including sequences of SNPs of known ancestral origins to generate predictions of ancestral origins of the sequences. A comparison between the predicted and true ancestral origin (or between the predicted and known geographical coordinates of ancestral origin locale) of each SNP segment can be made. Various parameters of the predictor sub-model and the smoothing sub-model, such as the weights of the fully-connected neural network model, the parameters of the kernel of the convolutional neural network model, the decision trees, the weights associated with the SNP segments in the smoothing operations, etc., can be adjusted to maximize a degree of matching between the predicted and true ancestral origins.

In a case where machine learning model 200 operates as a classifier to classify an SNP segment into one of candidate ancestral origins, machine learning model 200 can be trained based on a cross-entropy loss function. Cross-entropy generally refers to a measure of the difference between two probability distributions for a given random variable or set of events. Entropy is the number of bits required to transmit a randomly selected event from a probability distribution, whereas a cross-entropy calculates the number of bits required to represent or transmit an average event from one distribution compared to another distribution. The cross-entropy between a target distribution, P, and an approximation of the target distribution, Q, can be calculated using the probabilities of the events from P and Q, as follows:

H(P,Q)=−ΣₓxP(x)×log(Q(x)) (Equation 6)

In Equation 6, P(x) is the probability of the event x in P, whereas Q(x) is the probability of event x in Q.

Cross-entropy can be used as a loss function to optimize machine learning model 200 operating as a classifier. As explained above, machine learning model 200 can compute, for an SNP segment, a probability for each candidate ancestral origin. A cross-entropy loss function can be determined for that SNP segment based on the expected probability of each candidate ancestral origin in the training data (e.g., based on a distribution of the known ancestral origins in the simulated genomic sequence of simulated admixed descendants) and the predicted probability output by machine learning model 200 for each candidate ancestral origin, based on Equation 6. Referring to Equation 6, event x can be to the set of candidate ancestral origins (e.g., Africa, East Asia, Europe), P(x) can be expected probability of each candidate ancestral origin, whereas Q(x) can be the predicted probability output by the machine learning model for each candidate ancestral origin.

In some examples, the training operation can be based on a combined cross-entropy loss function, which can include a linear combination of a first cross-entropy loss function associated with predictor sub-model 206, and a second cross-entropy loss function associated with smoothing sub-model 208, as follows:

L(y, \hat{y})=λ₁ [symbol] _{CE1}(y, \hat{y}₁)+λ₂ [symbol] _{CE2}(y, \hat{y}₂) (Equation 7)

In Equation 7, [symbol] _{CE1}(y, ŷ₁) can include the first cross-entropy loss function associated with predictor sub-model 206. The first cross-entropy loss function [symbol] _{CE1}(y, ŷ₁) can compare the initial ancestral origin estimates ŷ₁ (e.g., predicted probabilities for each candidate ancestral origin) output by the predictor sub-model for segments of SNPs in training data with their true ancestral origins y (e.g., expected probabilities for each ancestral origin) to generate first loss gradients, which can be used to adjust the weights in the fully-connected neural network of predictor sub-model 206 to minimize the first cross-entropy loss function. Moreover, the second cross-entropy loss function [symbol] _{CE2}(y ŷ₁) can compare the final prediction results ŷ₂ (e.g., predicted probabilities for each candidate ancestral origin) output by the predictor sub-model for segments of SNPs in training data with their true ancestral origins y (e.g., expected probabilities for each ancestral origin) to generate second loss gradients, which can be used to adjust the kernel of CNN of smoothing sub-model 208. When λ₁>0, the output of predictor sub-model 206, ŷ₁, represents the probabilities estimated by the classifiers, otherwise the output of the classifiers can be interpreted as a hidden layer. In some examples, each of λ₁ and λ₂ can be set to ½. The neural networks of the overall machine learning model can also be trained using various optimizers, such as Adam optimizer, stochastic gradient descent (SGD), rmsprop, etc., and a learning rate of 0.01 over 100 epochs.

In addition, during the training operation the output of predictor sub-model 206 for each segment of SNPs in the training data can be used to determine the usefulness metric of the segment of the SNPs. As described above, as part of the weighted averaging operation, smoothing sub-model 208 can assign weights to each initial ancestral origin estimate based on usefulness metrics of the segment of the SNPs represented by the respective initial ancestral origin estimate. The usefulness metrics can reflect, for example, whether the ancestral origins of the SNP variants at certain SNP sites of the segment can be correctly predicted. The usefulness metrics can be based on, for example, a probability of prediction error of ancestral origin for the segment, which can be determined based on the prior prediction results of the segment of genomes of a population. Here, based on the first cross-entropy loss function, a probability of prediction error at predictor sub-model 206 can be determined for each segment as part of the usefulness metrics. The probability prediction error can be forwarded to smoothing sub-model 208 and can be combined with the outputs of the second cross-entropy loss function [symbol] _{CE2}(y, ŷ₁) to update the weights.

For the examples of neural networks above, the number of the parameters can be reduced to improve computation efficiency. Example techniques to reduce the number of parameters may include weight sharing, weight factorization, weight quantization, etc. In addition, multi-task systems that provide both classification and regression simultaneously implemented by extending the number of outputs of the system. The training can also be adapted to provide both classification and regression.

F. Training With Data From Dimensionality Reduction Operation

As described above, predictor sub-model 206 can be trained using training data obtained from a dimensionality reduction operation. Dimensionality reduction generally involves transformation of data from a high-dimensional space into a low-dimensional space. In the case of predictor sub-model 206, the low-dimensional representation can be used as the output labels that distinguish between different ancestral origins of input segment of SNPs. Examples of dimensionality reduction operation include, for example, principal component analysis (PCA), kernel PCA, autoencoder, T-distributed Stochastic Neighbor Embedding (t-SNE), uniform manifold approximation and projection (UMAP), etc.

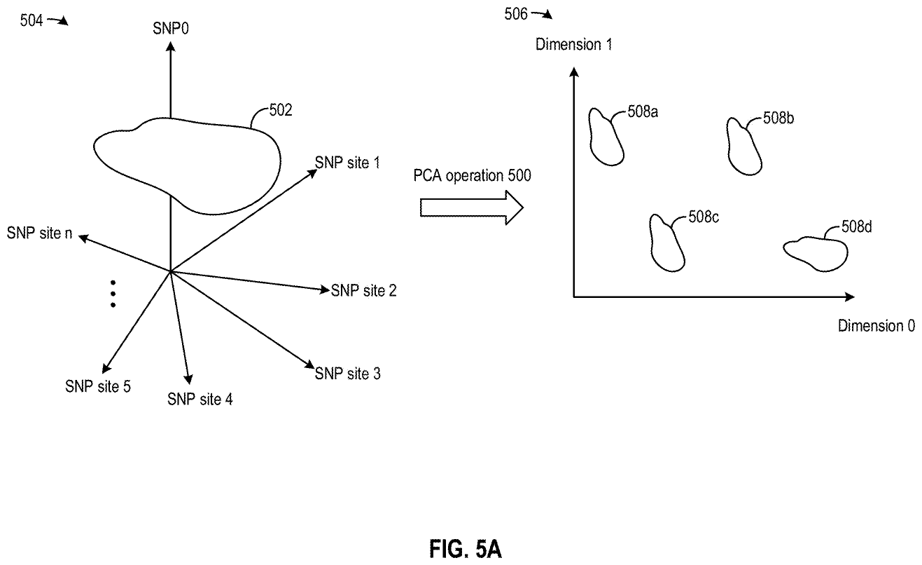

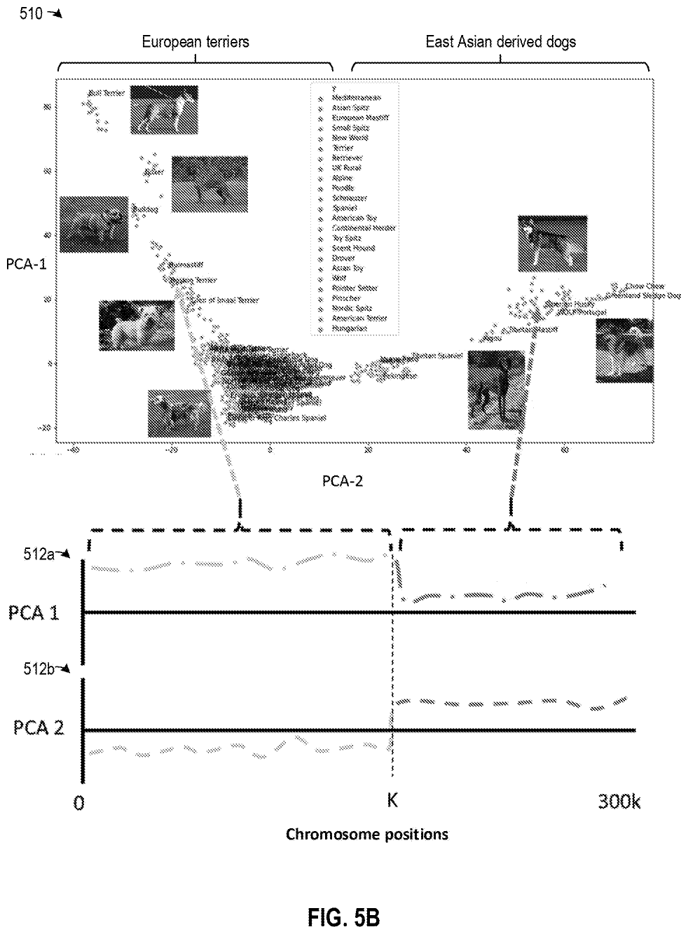

FIG. 5A illustrates an example of a principal component analysis (PCA) operation 500. As shown on the left of FIG. 5A, genomic sequences 502 of subjects can be represented in a high-dimensional space 504 with each dimension representing, for example, a particular SNP site (e.g., SNP site 0, SNP site 1, SNP site 2, . . . SNP site n). In a case where the genome has a million SNP sites, high-dimensional space 504 can have a million dimensions. A genomic sequence can have a coordinate (e.g., 0 for one allele and 1 for the other allele) at each dimension representing an SNP value for an SNP site represented by the dimension, and the coordinates at each dimension can form a vector of a million dimensions. When generating a training set for use with breed coordinates, the training samples (reference subjects) can be pure breeds with known ancestral origin.

As part of PCA operation 500, a linear transformation can be performed on the vectors representing genomic sequences 502 in high-dimensional space 504 to a low-dimension space 506, which can include two dimensions labelled dimension 1 and dimension 2. The transformation can be such that the greatest variance by some scalar projection of the vectors lie on dimension 1, which can be the first principle component, and the second greatest variance can lie on dimension 2, which can be the second principle component. In other examples, low-dimension space 506 can include more than two dimensions. The transformation can be represented by the following Equation:

tᵢ=zᵢ·w (Equation 8)

In Equation 8, zᵢ can be a vector representing a genomic sequence associated with a label i and with p dimensions defined in high-dimension space 504, whereas w can be a p-dimensional weight vector (w₁, w₂, wₚ). Moreover, a new vector tᵢ of principle component scores can be generated from a dot product between zᵢ and w. The principle component scores can also represent the coordinates of the vector zᵢ in low-dimension space 506.

Referring back to FIG. 3B and FIG. 3C, after the PCA operation is performed on the genomic sequences of a set of reference subjects and their coordinates in low-dimension space 506 are obtained, the genomic sequences of the set of reference subjects and their reference coordinates can be used to train the neural networks of FIG. 3A-FIG. 3D. As part of the training operation, the sets of weights W₀, W₁, . . . Wₙ₋₁ are updated to minimize the differences between the coordinates output by the neural networks (e.g., by output layers 316, 326, etc.) and the reference coordinates for each SNP segment of the genomic sequences of the set of reference subjects. The full genomic sequences of the reference subjects used in the training can have a known pure ancestral origin, race, breed, etc., such that all SNP sites of the genomic sequence carries an SNP value indicative of that pure ancestral origin, race, or breed, and the neural network can be trained such that the output coordinates for all SNP sites indicate the same ancestral origin, race, breed, etc. After the weights of a neural network is trained, segments of SNPs of a new subject can then be fed into the neural network to determine the coordinates of each segment in low-dimension space 506. The coordinates can reflect the ancestral origin/race/breed represented by that segment.

Through a PCA operation, a genomic sequence (or a segment of SNP sites) can be represented by a set of coordinates (breed coordinates) in a multi-dimensional space, such as low-dimension space 504. As the dimensions in low-dimension space 506 represent projection of vectors of high variances, these vectors can be encoded with the set of coordinates to highlight the differences in important features (e.g., patterns of SNP values at SNP sites) of the genomic sequences that distinguish between the ancestral origins of the subjects, and genomic sequences having differences in such important features can be separated out into clusters in low-dimension space 346. For example, as shown in FIG. 5A, genomic sequences 502 can be aggregated into clusters 508a, 508b, 508c, and 508d through their representations in low-dimension space 506. Each cluster can correspond to a different ancestral origin/race/breed. The coordinates of an SNP segment of a new subject can be compared with the coordinates of these clusters to predict the ancestral origin/race/breed represented by that segment.

FIG. 5B illustrate another example of a dimensionality reduction operation to supply training data to train machine learning model 200 of FIG. 2A-FIG. 2C. In FIG. 5B, the training data can be used to train machine learning model 200 to determine, at each chromosome position of an input genomic sequence, coordinates indicative of a breed or ancestral origin. For example, machine learning model 200 can be trained using full genomic sequences of pure-bred European terriers and East Asian derived dogs to generate coordinates for SNPs at each chromosome position/SNP site in a two-dimensional space with two dimensions, labelled PCA-1 and PCA-2. Each full genomic sequence may include about 1 million SNP sites. Other embodiments can use from about 10,000 to about ten million SNP sites. A full genomic sequence is represented by a pair of coordinates in the PCA-1 dimension and in the PCA-2 dimension. It is understood that the dimensionality reduction operation can generate more than two dimensions for the space (e.g., three dimensions or more). Moreover, SNPs are generally biallelic, and a vector representing a full genomic sequence can use (0.1) encoding or other encoding.

The top of FIG. 5B illustrates a graph 510 of a distribution of coordinates of the full genomic sequences of reference subjects generated by PCA, comprising pure-bred European terriers and East Asian derived dogs, in the two-dimensional space. As shown in the graphs, European terriers tend to have relatively high coordinate values (e.g., 0-80) along the PCA-1 dimension, and relatively low coordinates (e.g., −20-0) along the PCA-2 dimension. In contrast, East Asian derived dogs tend to have relatively high coordinate values along the PCA-2 dimension (e.g., 20-80) and relatively low coordinate values along the PCA-1 dimension (e.g., 0-20).

The bottom of FIG. 5B illustrates graphs 512a and 512b, which shows the coordinate values for each chromosome position of a new subject along the PCA-1 dimension and the PCA-2 dimension output by the trained machine learning model 200. The machine learning model 200 can be trained using the full genome sequences of reference subjects and their reference coordinates along the PCA-1 and PCA-2 dimensions shown in graph 510. As shown in graphs 512a and 512b, the coordinate values for a first region of chromosome between chromosome positions 0 to K may have relatively high coordinate values in the PCA-1 dimension and relatively low coordinate values in the PCA-2 dimension, which can indicate the first region of chromosome may come from European terriers. Moreover, a second region of chromosome, from K to 300k, may have relatively low coordinate values in the PCA-1 dimension and relatively high coordinate values in the PCA-1 dimension, which can indicate that the second region of the chromosome may come from East Asian derived dog. In particular, each region (or sliding window) can be mapped to particular values for PCA-1 and PCA-2, which can then be compared to the known coordinates of breeds. A distances between the coordinates of a new test subject and the reference subjects can provide a level of similarity for the given region.

G. Decision Trees As Prediction and Smoothing Sub-Models

Besides neural network, predictor sub-model 206 and smoothing sub-model 208 can be implemented using other techniques, such as decision trees. Compared with a neural network, the training and execution of decision trees can be less computation intensive and allow for more parallel executions, which allow a machine learning model built using decision trees to be executed and trained on various hardware platforms, including those with less computation resources and/or lower bandwidth. This can further improve the accessibility of local-ancestry inference operations. In some examples, a combination of neural networks and decision trees can be implemented in both predictor sub-model 206 and smoothing sub-model 208.

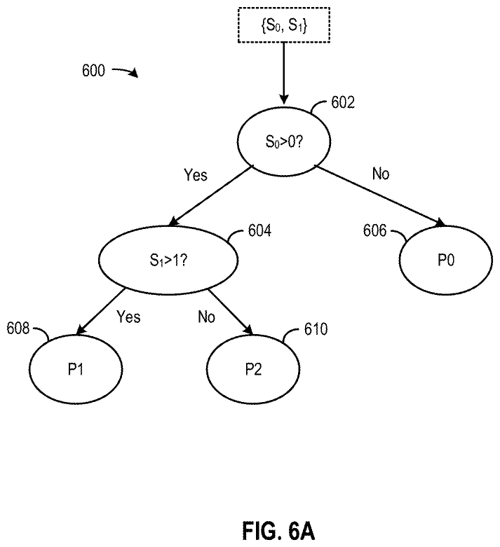

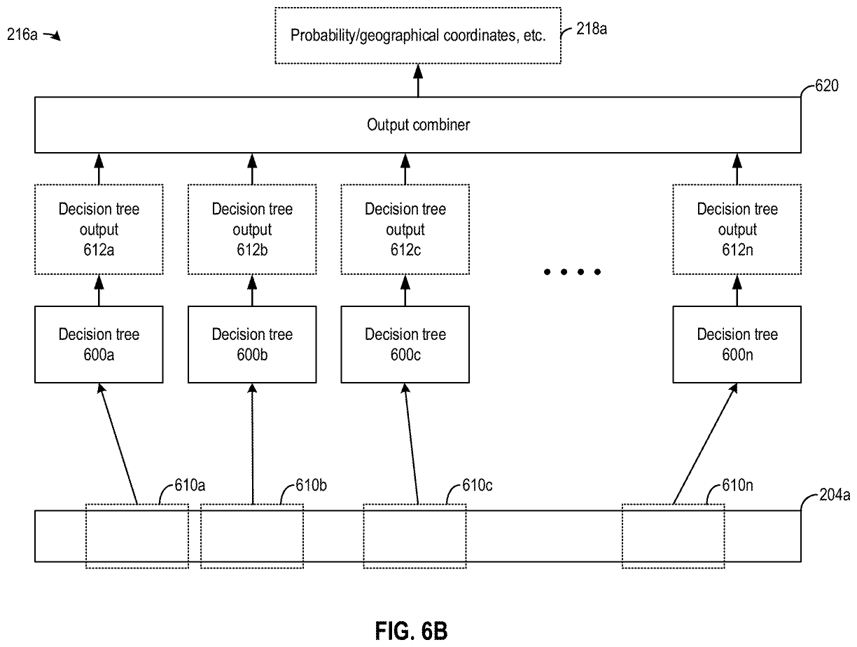

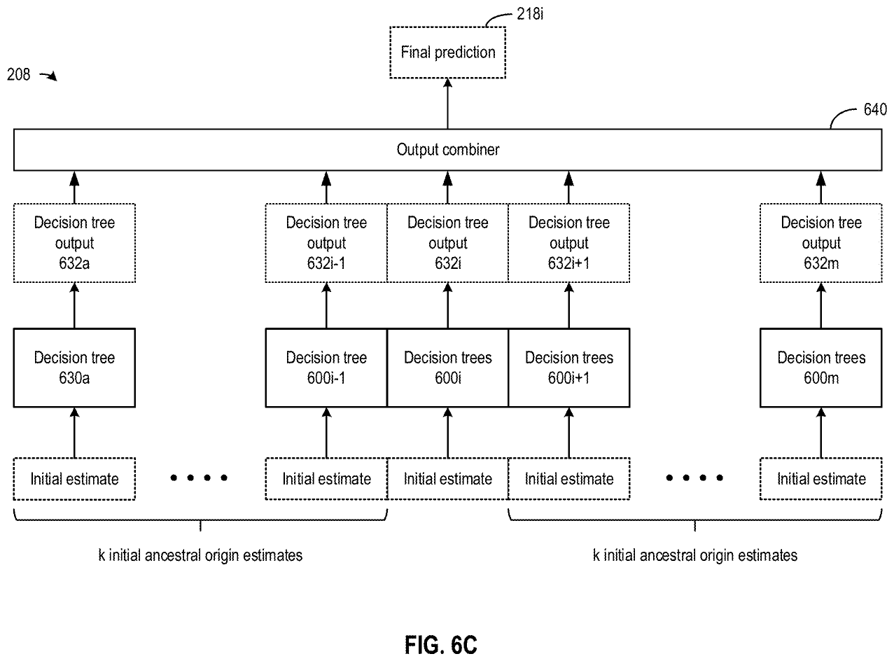

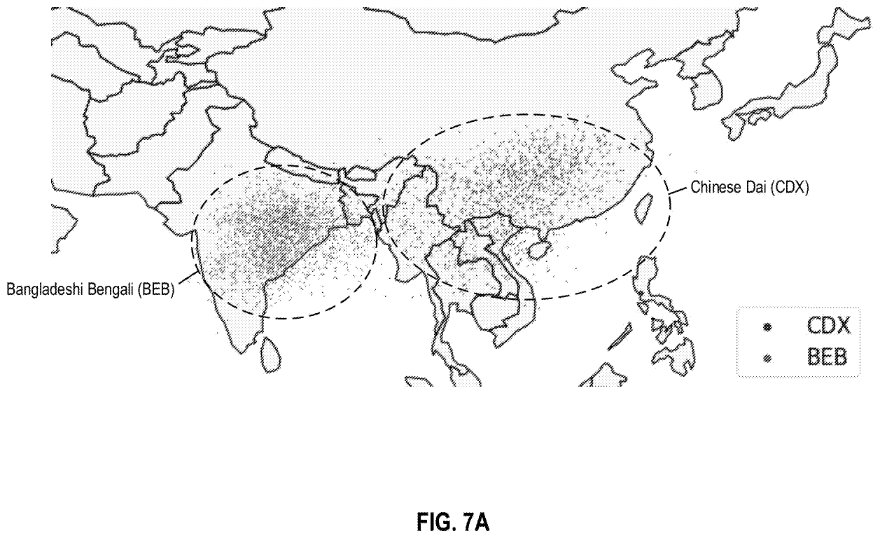

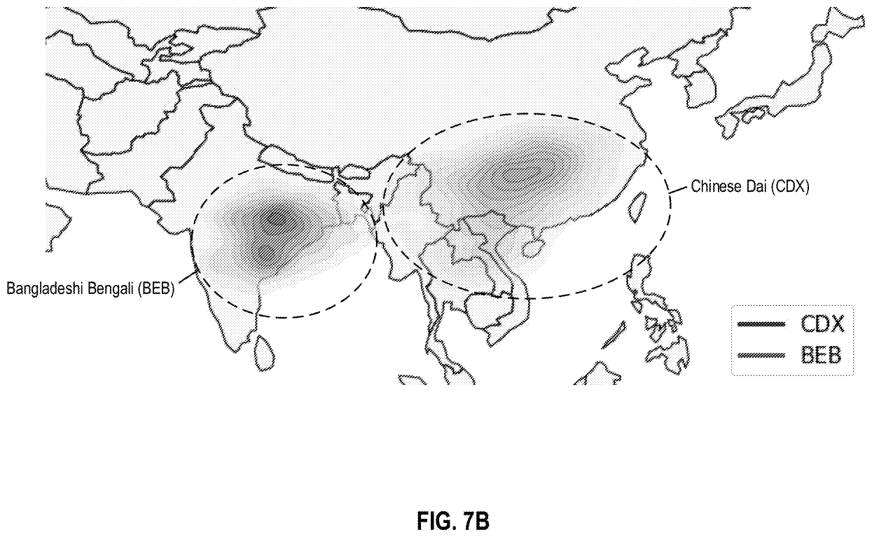

FIG. 6A illustrates examples of a decision tree 600, which can be configured to generate a decision regarding an input SNP sequence {s₀, s₁}. The decision can include, for example, the probability of the input SNP sequence having a particular ancestral origin, the geographical coordinates of the ancestral origin of the input SNP sequence, etc. One example of decision tree may include, for example, XGBoost trees.

As shown in FIG. 6A, a decision tree 600 can include a root node, such as root node 602, as well as child nodes, such as child nodes 604, 606, 608, and 610. Each parent node that has child nodes (e.g., nodes 602 and 604) can be associated with pre-determined classification criteria (e.g., a threshold for s₀, s₁, or a combination of both) to classify the input SNP sequence into a child node. Child nodes that do not have child nodes are terminal nodes. The terminal nodes include nodes 606, 608, and 610, each being associated with a decision output by a decision tree. In the example of FIG. 6A, decision tree 600 can have a depth of 3. Decision tree 600 can process a sequence of two SNPs and generate the probability of the input SNP sequence having a particular ancestral origin (e.g., one of Africa, East Asia, or Europe), and each of nodes 606, 608, and 610 is associated, respectively, with probabilities P0, P1, and P2. Based on the combination of criteria in parent nodes, decision tree 600 can output different probabilities for different patterns of input SNP sequences. Notice that decision tree 600 is provided as an illustrative example. It is understood that a decision tree used in predictor sub-model 206 can have a different number of nodes, different depths, and process a different number of SNPs in a sequence.

Referring back to FIG. 2A, a predictor unit, such as predictor unit 216a, can include multiple decision trees. Each decision tree can be assigned to process different subsets of an SNP segment, and the decisions output by the decision trees can be combined to generate an initial ancestral origin estimate, which can include the probabilities of the SNP segment being classified into each candidate ancestral origin, the geographical coordinates of the ancestral origin locale of the SNP segment, etc.

FIG. 6B illustrates an example of predictor unit 216a implemented based on decision trees. As shown in FIG. 6B, predictor unit 216a includes a plurality of decision trees including decision trees 600a, 600b, 600c, 600n, etc. Each decision tree can have different tree structures (e.g., different number of parent nodes and child nodes, different depths, etc.), as well as different decision criteria. Each decision tree can be assigned to process a subset of SNPs of input segment 204a. Decision tree 600a can be assigned to process subset 610a to generate a decision tree output 612a, decision tree 600b can be assigned to process subset 610b to generate a decision tree output 612b, decision tree 600c can be assigned to process subset 610c to generate a decision tree output 612c, whereas decision tree 600n can be assigned to process subset 610n to generate a decision tree output 612n. Each of decision trees 600a-n can have different tree structures, different classification criteria, etc. Moreover, different predictor units can also have different numbers of decision trees, and the decision trees can have different tree structures and classification criteria between different predictor units.

Predictor unit 216a further includes an output combiner 620 to combine the decision tree outputs into initial ancestral origin estimate 218a. In some examples, output combiner 620 can generate initial ancestral origin estimate 218a based on, for example, averaging/summing the decision tree outputs by the decision trees to generate a probability estimate. In some examples, predictor unit 216a can be configured as a regressor, and output combiner 620 can perform an weighted sum of the decision tree outputs based on the regression model parameters to generate the geographical coordinates of an ancestral origin locale for the input segment.

Besides predictor sub-model 206, smoothing sub-model 208 can also be implemented based on decision trees. FIG. 6C illustrates an example of smoothing sub-model 208 including a plurality of decision trees 630. Each decision tree 630 (e.g., decision trees 630a, 630m, etc.) can have a similar structure (e.g., including parent and child nodes) as decision tree 600 shown in FIG. 6A. Each decision tree can be assigned to process an initial ancestral origin estimate 218, and generate a decision tree output 632. Each decision tree output can represent a weighted version of the input ancestral origin estimate 218, with each weight representing a weight in a kernel (e.g., kernel 402 of FIG. 4A). Smoothing sub-model 208 further includes an output combiner 640 to combine the decision tree outputs. Output combiner 640 can, for example, sum the decision tree outputs to generate final prediction results 220.

Decision trees 630, together with output combiner 640, can perform the smoothing function based on a sliding window as in FIG. 4A. For example, as shown in FIG. 6C, decision trees 630 can be assigned to process a set of initial ancestral origin estimates included in a window including initial ancestral origin estimates 218a to 218i−1, initial ancestral origin estimate 218i, and initial ancestral origin estimates 218i+1 to 218m, to generate a final prediction result 220i which is to replace the target initial ancestral origin estimate 218i. The window can be configured such that it centers around target initial ancestral origin estimate 218i. For example, the window can include k (e.g., 50) initial ancestral origin estimates before and after target initial ancestral origin estimate 218i. For the next final prediction result, a different window of initial ancestral origins can be input to decision trees 630 to generate the final prediction result.

H. Training of Decision Trees Sub-Models

The decision trees of predictor sub-model 206 and smoothing sub-model 208 can be trained using training data derived from full genome data of the population of known ancestral origins, including individuals from various locales of Africa, East Asia, and Europe. From the full genome sequence of these individuals, simulated genome sequence of simulated admixed descendants of these individuals is generated based on Wright-Fisher forward simulation over a series of generations. A set of training data comprising a genome sequence of simulated admixed descendants of these individuals with a wide range of generations, as well as the known ancestral origins of SNP segments of the simulated genome sequences, can be used to train and validate the machine learning model, which allows the machine learning model to learn from the relationships between patterns of SNP variants at different DNA sites and their ancestral origins reflected in the training data to perform local-ancestry inference.

The decision trees of predictor sub-model 206 and smoothing sub-model 208 can be trained based on a gradient tree boosting operation. Specifically, the training operation can start with creating a first decision tree to fit the first decision outputs (e.g., ancestral origin estimates, geographical coordinates of ancestral origin locales, etc.) with segments of SNPs of training data. A first set of residuals, which can represent the differences between the first decision output of the first decision tree and the ground truth can be determined. A first regression relationship between the ground truth/target ancestral origins and the SNP segments, provided by the first decision outputs of the first decision tree, can be as follows:

Y=f1(x) (Equation 9)

In Equation 9, Y is the ground truth/target ancestral origins, whereas f1(x) represents a regression model that relates the SNP segments in the training data to Y. A first set of residuals, which represent the differences between the ground truth/target ancestral origins and the regression estimates by the first decision tree, can be as follows:

First_Residual(x)=Y−f1(x) (Equation 10)

A second decision tree can then be generated and trained to fit the second decision output over the first set of residuals. For example, the second decision tree can be trained to generate second decision output that matches the first set of residuals as much as possible, for the same segment of SNPs input to the first decision tree. A second regression relationship between the first set of residuals and the SNP segment, provided by the second decision output of the second decision tree, can be as follows:

Y−f1(x)=f2(x) (Equation 11)

A second set of residuals, which represent the differences between the first set of residuals and the regression estimates by the second decision tree, can be as follows:

Second_Residual(x)=Y−f1(x)−f2(x) (Equation 12)

A third decision tree can then be generated and trained to fit the third decision output over the second set of residuals. The training process can be repeated until, for example, a pre-determined number of trees is reached, a pre-determined threshold level of residuals is achieved, etc. Through the addition of new decision trees to fit the decision tree outputs with the residuals, the decision trees can represent a regression model of a relationship between SNPs and ancestral origin estimates and/or geographical coordinates of ancestral origin locale, as follows:

Y=f1(x)+f 2(x)+ . . . +fn(x) (Equation 13)

The decision trees in predictor sub-model 206 and smoothing sub-model 208 can be trained separately in separate gradient tree boosting operations and can have different learning rate. For example, predictor sub-model 208 can be trained based on a learning rate of 0.1, whereas smoothing sub-model 208 can be trained based on a learning rate of 0.3.

Compared with a neural network, the training and execution of decision trees can be less computation intensive and allow for more parallel executions, which allow a machine learning model built using decision trees to be executed and trained on various hardware platforms, including those with less computation resources and/or lower bandwidth. This can further improve the accessibility of local-ancestry inference operations. In addition, the robustness of the machine learning model can be improved when the model is trained as a regressor to estimate geographical coordinates of ancestral origin locales of the segments of SNPs based on a regression operation, which can provide useful ancestral estimates even for closed related populations.

In some examples, the decision trees shown in FIG. 6A-FIG. 6C can also be trained as a regressor to generate coordinates, or codes, to represent an ancestral origin/breed, as described above in FIG. 5A-FIG. 5B. The decision trees can be trained using vectors representing full genomic sequence of pure breed subjects, or subjects having a single ancestral origin for all SNP segments, and reference coordinates in the multi-dimensional space obtained via a dimensionality reduction operation on the vectors.

### II. Experimental Results

A. Local-ancestry Inference Based on Neural Networks

An example of machine learning model 200 of FIG. 3A-3C, comprising a fully-connected neural network with a hidden layer as predictor sub-model 206, as well as a convolutional neural network as smoothing sub-model 208, is developed and trained. The training data is derived from full genome sequences of a total of 1668 single-population individuals from East Asia (EAS), African (AFR) and European (EUR) ancestry. The East Asian group is composed of the following individuals: 103 Han Chinese in Beijing, China (CHB), 104 Japanese in Tokyo, Japan (JPT), 105 Southern Han Chinese (CHS), 93Chinese Dai in Xishuangbanna, China (CDX) and 99 Kinh in Ho Chi Minh City, Vietnam (KHV). The African group is composed of the following individuals: 108 Yoruba in Ibadan, Nigeria (YRI), 99 Luhya in Webuye, Kenya (LWK), 113 Gambian in Western Divisions in the Gambia (GWD), 85 Mende in Sierra Leone (MSL), 99 Esan in Nigeria (ESN), 61 Americans of African Ancestry in Southwest USA (ASW) and 96 African Caribbeans in Barbados (ACB). Finally, the European group is composed of the following sub-populations: 99 Utah Residents (CEPH) with Northern and Western European Ancestry (CEU), 107 Toscani in Italia (TSI), 99 Finnish in Finland (FIN), 91 British in England and Scotland (GBR) and 107 Iberian Population in Spain (IBS).

Using the full genomes of these individuals, genome data of simulated admixed descendants are obtained using Wright-Fisher forward simulation over a series of generations. In particular, from the 1668 single-population individuals, 1328 were selected to generate 600 admixed individuals for training, 170 were used to generate 400 admixed individuals for validation and the remaining 170 were used to generate 400 admixed individuals for testing. The validation and testing set was generated using 10 individuals for each of the 17 different ancestries. The 600 admixed individuals of the training set were composed by groups of 100 individuals generated after 2, 4, 16, 32 and 64 generations. The 400 admixed individuals of the validation and testing set were generated with 6, 12, 24 and 48 generations each.

The genome data are divided into a training data set, a validation data set, and a test data set. The entire machine learning model (including predictor sub-model 206 and smoothing sub-model 208) is trained using the training data set and based on the combined cross-entropy loss function of Equation 7. Moreover, various hyper parameters of the machine learning model, such as the parameters of the combined cross-entropy loss function (e.g., λ₁ and λ₂), the number of SNP sequences processed by a predictor unit, the number of initial ancestral origin estimates included in a window, the hidden layer size, the smoothing kernel size, etc., can be determined from the validation data set. The test data set is then used to test the machine learning model after training and with hyper parameters updated based on the validation data.

1. Test and Validation Results

Table 1 below presents the accuracy results for chromosome 20 of the examples of machine learning model 200 of FIG. 3A and FIG. 3B (no hidden layer) and of FIG. 3C (with hidden layer), with and without smoothing operations.

TABLE 1

| Method | | Validation | Test |
|---|---|---|---|
| Neural net without | Without smoothing | 79.70% | 77.78% |
| hidden layer | With smoothing | 97.10% | 96.85% |
| Neural net with | Without smoothing | 82.20% | 80.29% |
| hidden layer | With smoothing | 97.96% | 97.85% |

Table 1 above suggests that machine learning model 200 based on neural networks can achieve state-of-the-art performance. With only two and three layers, the model size of the networks are about 10 Mb (without hidden layer) and about 100 Mb (with hidden layer). Both models are trained here with data from chromosome 20, and their sizes can scale linearly with larger chromosomes.

2. Missing Data Robustness

Applications that work with genotype data commonly face data that is noisy or incomplete due to genotyping errors. In other cases only a subset of SNPs might be available due to differing commercial genotyping arrays. Therefore, robustness to missing data is an important element. To improve robustness of machine learning model 200 in handling missing data, the machine learning model can be trained and tested with different percentages of missing input SNPs. The structure of the network was not changed and the missing labels were modeled by applying dropout to the input data in both training and testing (i.e. missing SNPs were set to 0).

Table 2 below presents the accuracy values of the ancestral origin estimates by machine learning model 200 of FIG. 3A-3C with a different percentage of missing input SNPs, with and without smoothing sub-model 208.

TABLE 2

| % Missing SNPs | w/out Smoothing | w/Smoothing |
|---|---|---|
| 0 | 80.29% | 97.85% |
| 25 | 68.16% | 95.70% |
| 50 | 62.55% | 94.01% |
| 75 | 55.82% | 92.36% |
| 90 | 48.36% | 87.06% |

The accuracy results suggest that the network is able to accurately infer ancestry without a considerable loss of accuracy, even when 50% of the input SNPs are missing. Another advantage is that if only 50% of the input SNPs are used during deployment, only half of the model parameters need to be stored and only half of the data needs to be processed. This turns missing data from an annoyance into a feature for designing smaller and faster networks that require a fraction of the number of input SNPs as an input.

3. Phasing Error Robustness

Humans carry two complete copies of the genome, one from each parent. Current sequencing technologies are typically unable to ascertain whether two neighboring SNP variants belong to the same sequence (maternal or paternal) or opposite sequence. That is, read base-pairs cannot be properly assigned to the paternal or maternal sequences. Assigning variants to their correct sequence is known as phasing, and statistical algorithms have been developed to solve this problem based on observed correlations between neighboring SNP variants allele in reference populations.

Examples of machine learning model 200 of FIGS. 3A-3C can be trained and tested with data containing different percentages of phasing errors. In order to model these errors, the genomic sequence are randomly swapped in locations where the base-pairs differed in the maternal and paternal sequences. In other words, after encoding the SNPs as −1 and 1, the sign of the SNPs in positions where the paternal and maternal are 1 and −1 or vise-versa, were switched with a probability p.

Table 3 presents the accuracy results of machine learning model 200, with and without smoothing sub-model 208, when different values of p were used for training and evaluation. Results suggest that the network is able to handle small and medium levels of phasing errors, however the accuracy decreases considerably when very high phasing errors (˜40%) are present.

TABLE 3

| % Phasing Errors | w/out Smoothing | w/Smoothing |
|---|---|---|
| 0 | 80.29% | 97.85% |
| 5 | 78.61% | 97.75% |
| 10 | 76.94% | 97.52% |
| 20 | 72.98% | 96.85% |
| 30 | 68.18% | 95.59% |
| 40 | 60.64% | 88.89% |

B. Local-Ancestry Inference Based on Decision Trees

An example of machine learning model 200 of FIG. 5A-5C, comprising decision trees in predictor sub-model 206 and smoothing sub-model 208, is developed and trained. The machine learning model can be implemented based on XGBoost system. The training data is derived from full genome sequences of a total of 318 single-population individuals from East Asia (EAS), African (AFR) and European (EUR) ancestry. The African group consisted of 108 Yoruba from Ibadan, Nigeria (YRI), the East Asian group of 103 Han Chinese from Beijing, China (CHB), and the European group of 107 Spanish individuals (IBS).

Using the full genomes of these individuals, genome data of simulated admixed descendants are obtained using Wright-Fisher forward simulation over a series of generations. In particular, from the 318 single-population individuals, 258 were selected to generate 600 admixed individuals for training. Ten individuals were selected to generate 300 admixed individuals for validation and the remaining ten were selected to generate 300 admixed individuals for testing. The training set, composed of 600 admixed individuals, consists of 6 groups of 100 individuals generated by 2, 8, 12, 32, 48 and 64 generations. The validation and testing set, composed of 300 admixed individuals each, consist of three groups of 100 individuals generated by 4, 16, and 24 generations.

Additionally, a dataset with closely located (and genetically similar) populations to perform a qualitative evaluation of the method's performance when faced with challenging admixed individuals. 400 simulated admixed individuals are generated using populations located across Asia including: 182 Han Chinese (CHB and CHS), 83 Chinese Dai (CDX), 89 Vietnamese Kinh (KHV), 94 Japanese (JPT), 93 Gujarati Indians (GIH), 86 Pakistani Punjabi (PJL), 76 Bangladeshi Bengali (BEB), 92 Sri Lankan Tamil (STU) and 92 Indian Telugu (ITU). A total of 10 individuals per population were used to generate 200 individuals for testing. The rest of the individuals were used to generate 200 admixed individuals for training. Both training and testing individuals were generated after 2 and 4 generations. Since local-ancestry inference methods must accurately estimate the ancestry from individuals regardless of their admixture histories (different generation times since admixture), it is important to train and evaluate the method with admixed individuals simulated over a wide range of generations.

The genome data are divided into a training data set, a validation data set, and a test data set. The entire machine learning model (including predictor sub-model 206 and smoothing sub-model 208) is trained using the training data set based on gradient tree boosting operation as described above. Moreover, various hyper parameters of the machine learning model, such as the a number of SNPs processed by a decision tree, a number of decision trees included in each predictor unit, levels of trees, the learning rate, etc., can be based on the validation data set. The test data set is then used to test the machine learning model after training and with hyper parameters updated based on the validation data.

1. Test and Validation Results

Table 4 below presents the accuracy results for chromosome 20 of the examples of machine learning model 200 of FIGS. 6A-6C trained as a classification model and a regression model, with and without smoothing operations.

TABLE 4

| | | |
|---|---|---|
| Model | Validation | Test |

| | | | |
|---|---|---|---|
| Classifier | Without smoothing | 78.30% | 78.59% |
| | With smoothing | 98.20% | 97.98% |
| Regressor | Without smoothing | 78.30% | 78.34% |
| | With smoothing | 96.63% | 96.59% |

Tests suggest that both the decision-tree-based machine learning model, configured as a classification model or a regression model, can achieve state-of-the-art accuracy with no significant difference between the classification and regression models.

2. Missing Data Robustness

The examples of machine learning model 200 of FIGS. 6A-6C are also tested for their performances in handling missing SNP data. Genotype data can be incomplete due to genotyping errors, or only a subset of SNPs might be available depending on the commercial genotyping array used. Therefore, methods that are robust to missing SNP data are preferred. Table 5 below presents the prediction accuracy of machine learning model 200 of FIGS. 6A-6C trained as a classifier with a different percentage of missing input SNPs.

TABLE 5

| % Missing SNPs | w/out Smoothing | w/Smoothing |
|---|---|---|
| 0 | 78.59% | 97.98% |
| 20 | 74.88% | 96.78% |
| 50 | 69.20% | 95.00% |
| 80 | 58.78% | 93.19% |

Table 5 above suggest that the machine learning model is able to estimate ancestry without a significant loss of accuracy, even when 80% of the input SNPs are missing. This also enables the development of lightweight and fast methods that use only a small fraction (e.g. 20%) of the input data in cases where deployment time and efficiency are paramount.

3. Ancestral Origin Estimation for Closely-Related Animal Population and Crops

A qualitative evaluation of the decision-tree-based machine learning model 200, configured as a regression model to determine geographical coordinates of ancestral origins of SNP segments, is also performed. While classification-based approaches fail in closely related populations (obtaining ˜15% accuracy in this dataset), co-ordinates regression-based models are able to provide meaningful representations of an individual's ancestry.

FIG. 6A and FIG. 6B illustrate examples of estimated density maps of dual-ancestry admixed individuals using machine learning model 200 trained on all of the Asian populations, in the form of a point cloud map and a contour map respectively. Similar density maps of crops/animals having multiple ancestral origins can also be obtained using machine learning model 200.

4. Extended Experimental Results

In addition, various effects on the prediction accuracy of decision-tree-based machine learning model 200, such as a number of SNPs processed by each decision tree, the smoothing window size, and the generation times since simulated admixture, are studied.

Table 6 below illustrates the effect of the smoothing window size on individuals from different numbers of generations following admixture. Results show that larger smoothing window size provides better accuracy, except for individuals with large generation values following admixture. This can be because individuals for whom the admixing process happened many generations ago will have many ancestry switches. As a result, these individuals only have small fragments of SNPs for which ancestry origin remains constant. In such scenarios, information from distant windows isn't useful and larger smoothing window sizes may not improve prediction accuracy. On the other hand, for individuals whose admixing processes happen recently, ancestry switch frequency is typically low, and distant genomic regions may still be informative.

TABLE 6

| Smoothing | | | | | | | | |
|---|---|---|---|---|---|---|---|---|
| window | Generation Number | | | | | | | |
| size | 2 | 4 | 8 | 12 | 24 | 32 | 48 | 64 |
| No | 78.0 | 79.0 | 78.6 | 79.0 | 78.5 | 77.7 | 78.3 | 77.9 |
| smoothing | | | | | | | | |
| 20 | 97.2 | 97.6 | 97.0 | 96.7 | 96.1 | 95.8 | 95.5 | 94.6 |
| 50 | 98.6 | 99.0 | 98.3 | 98.1 | 97.5 | 96.5 | 95.7 | 94.8 |
| 100 | 99.1 | 99.3 | 98.8 | 98.4 | 97.6 | 96.5 | 95.2 | 93.9 |
| 200 | 99.3 | 99.5 | 98.9 | 98.4 | 97.6 | 96.4 | 95.2 | 93.9 |

Table 7 below shows the effect of including smoothing in relation to the number of SNPs processed by each predictor unit in a window. We can observe that for small window sizes (500 SNPs), the accuracy difference with and without smoothing is quite large (˜7%). However, when using large window sizes (2000 SNPs), the accuracy difference is lower (˜8%). The differences in the accuracy can be due to, for example, a larger window size being able to capture relationships between SNPs further away.

TABLE 7

| | Smoothing | | | | | | | | |
|---|---|---|---|---|---|---|---|---|---|
| Window | window | Generation Number | | | | | | | |
| size | size | 2 | 4 | 8 | 12 | 24 | 32 | 48 | 64 |
| 500 | No | 78.0 | 79.0 | 78.6 | 79.0 | 78.5 | 77.7 | 78.3 | 77.9 |
| | smoothing | | | | | | | | |
| | 50 | 98.6 | 99.0 | 98.3 | 98.1 | 97.5 | 96.5 | 95.7 | 94.8 |
| 1000 | No | 84.7 | 86.0 | 85.3 | 85.5 | 85.0 | 84.4 | 84.9 | 84.3 |
| | smoothing | | | | | | | | |
| | 50 | 98.9 | 99.3 | 98.6 | 98.5 | 98.0 | 97.2 | 96.6 | 95.7 |
| 2000 | No | 89.5 | 90.0 | 90.0 | 90.0 | 89.5 | 89.1 | 89.6 | 88.8 |
| | smoothing | | | | | | | | |
| | 50 | 98.3 | 98.6 | 98.0 | 97.8 | 97.4 | 96.5 | 96.5 | 95.7 |

A common behavior that can be observed in tables 6 and 7 is that the accuracy decreases as the number of generations following the admixing process increases. This can be because as a larger generation number implies more ancestry switches and therefore shorter sequences with constant ancestry origin, the switches become more challenging to detect.

Table 8 illustrates the mean absolute error of decision-tree-based machine learning model 200 in both admixed simulated datasets. The absolute error is measured in terms of the errors in the geographical coordinates (latitude and longitude) output by the model. Although the geographical distance within ancestries is larger in the continental dataset Africa/East Asia/Europe (“AFR-EUR-EAS”), the average error is lower because the method is able to properly discriminated between the three divergent ancestries. Within the Asia dataset, related (within nation) ancestries are very challenging to discriminate via a local ancestry approach, leading to a higher average error.

TABLE 8

| Dataset | Smoothing | Latitude error | Longitude error |
|---|---|---|---|
| Continental | No | 4.48 | 26.25 |
| (AFR-EUR-EAS) | Yes | 1.18 | 8.46 |
| Asian | No | 9.14 | 15.01 |
| (10 Classes) | Yes | 8.18 | 8.63 |

### III. Method

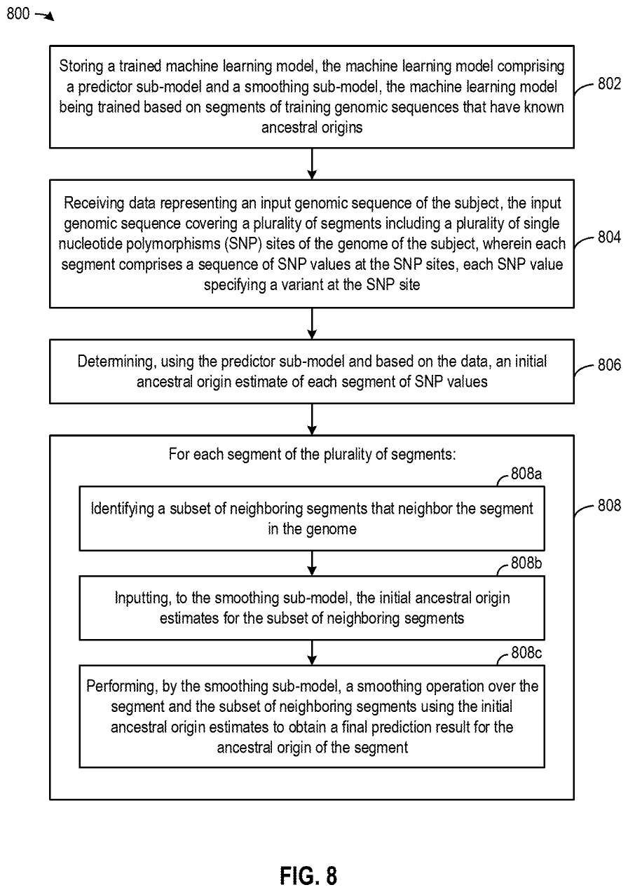

FIG. 8 illustrates a flowchart of a method 800 for determining an ancestral origin for different parts of a genome of a subject (e.g., a person). Method 800 can be implemented by a computer.

FIG. 8 starts with step 802, in which the computer stores a trained machine learning model, such as machine learning model 200 of FIG. 2A, in a memory of the computer. The machine learning model comprises predictor sub-model (e.g., predictor sub-model 206) and a smoothing sub-model (e.g., smoothing sub-model 208). The predictor sub-model may comprise, for example, a plurality of classifiers, a plurality of regressors, etc. A classifier can identify a probability (including binary 0 and 1) that a segment is from a particular ancestral origin; such a probability can be determined for each of a predetermined list of candidate ancestral origin categories. The initial ancestral origin estimate for the segment can be determined as the candidate ancestral origin category having the highest probability. Moreover, a regressor can provide a prediction that maps to geographical coordinates, or other types of identifiers, e.g., for providing accurate results within particular locales that are near each other. Each classifier and regressor can be implemented based on a neural network, as shown in FIG. 3A-FIG. 3C, or a set of decision trees, as shown in FIG. 6A-FIG. 6C.

The machine learning model can be trained based on segments of training genomic sequences that have known ancestral origins. The machine learning model can be trained based on training data derived from full genome data of a population of known ancestral origins to be identified by the machine learning model. For example, in a case where the machine learning model is to classify a segment into one of Africa, East Asia, and Europe, the training data can include genome data of individuals from various locales of Africa, East Asia, and Europe, as well as smaller geographic regions. From the full genome sequence of these individuals, simulated genome sequences of simulated admixed descendants of these individuals can be generated based on a simulation (e.g., a Wright-Fisher forward simulation) over a series of generations. A set of training data comprising genome sequences of simulated admixed descendants of these individuals (e.g., over numerous generations), as well as the known ancestral origins of segments of the simulated genome sequences, can be used to train and validate the machine learning model. The training can be based on, for example, a combined cross-entropy loss function, a gradient tree boosting operation, etc.

In step 804, the computer receives data representing an input genomic sequence of the subject, the input genomic sequence covering a plurality of segments including a plurality of single nucleotide polymorphisms (SNP) sites of the genome of the subject, wherein each segment comprises a sequence of SNP values at the SNP sites, each SNP value specifying a variant at the SNP site. Specifically, the data can be obtained from a haploid or diploid DNA sequence. The data can be obtained from, for example, a genome sequencing operation that provides a genome sequence of the subject, a DNA microarray that contains segments of DNAs, etc. The haplotype information in the data can be encoded to include, for example, different values for different variants. A first value can representing that the subject has a common variant (e.g., a value of −1) at a SNP site. A second value can represent that the subject has a minority variant (e.g., a value of +1) at the SNP site. A third value (e.g., a value of 0) can represent that the genomic information is missing at the SNP site.

In step 806, the computer determines, using the predictor sub-model and based on the data, an initial ancestral origin estimate of each segment of SNP values. Specifically, in some examples, each classifier can perform a classification operation on an non-overlapping segment of the SNPs to generate a classifier output. Each classifier can determine a probability of the segment being classified into each candidate ancestral origin categories (e.g., Africa, East Asia, and Europe), and the probabilities output by the classifiers can be combined to output an initial ancestral origin estimate based on the candidate ancestral origin category having the highest probability.

Moreover, in some examples, each regressor can perform a regression operation on a random subset of SNPs of the segment of the SNPs, which can be combined to output one or more origin estimates indicative of an ancestral origin of the segment of the SNPs. The one or more origin estimates can include, for example, the geographical coordinates (e.g., longitude and latitude) of the ancestral origin locale, a code representing the ancestral origin locale, etc. Further, in some examples, the regressor can also be trained to output coordinates in a multi-dimensional space obtained from a dimensionality reduction operation, with the coordinates representing an ancestral origin/breed of the subject.

In step 808, the computer can smooth the initial ancestral origin estimates to generate a final prediction result of an ancestral origin for each segment. For each segment of the plurality of segments, the computer can identify a subset of neighboring segments that neighboring the segment in the genome, in step 808a. The identification can be based on a sliding window which moves with a target initial ancestral origin estimate to be replaced by the final prediction result, as shown in FIG. 4A and FIG. 6C. The computer can then input the initial ancestral origin estimates for the subset of neighboring segments to the smoothing sub-model, in step 808b. The computer can then perform, using the smoothing sub-model, a smoothing operation over the segment and the subset of neighboring segments using the initial ancestral origin estimates to obtain the final prediction result for the ancestral origin of the segment, in step 808c. The smoothing operation can include computing a weighted average of initial ancestral origin estimates within a window. In some examples, a weight can be assigned to each initial ancestral origin based on a usefulness metric of the segment from which the initial ancestral origin is determined. The smoothing sub-model can include, for example, a convolutional neural network, a set of decision trees, etc.

### IV. Computer System

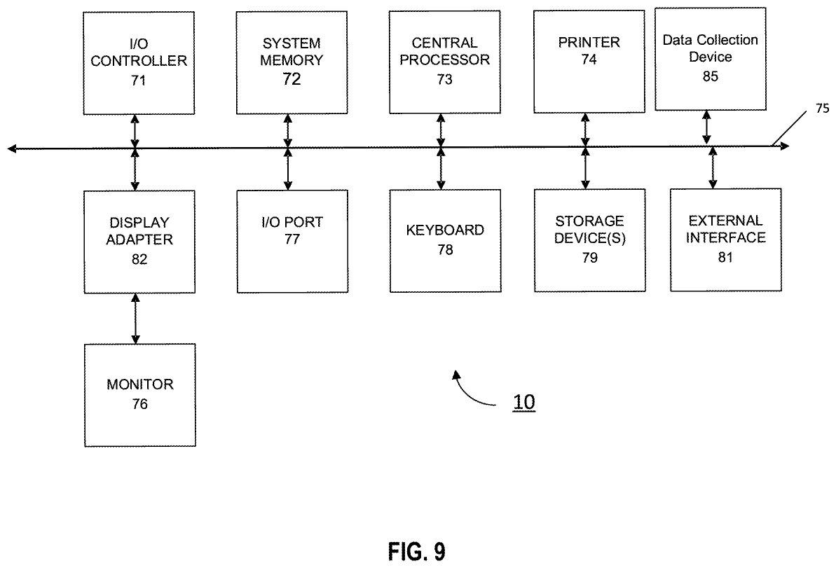

Any of the computer systems mentioned herein may utilize any suitable number of subsystems. Examples of such subsystems are shown in FIG. 9 in computer system 10. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices. In some embodiments, a cloud infrastructure (e.g., Amazon Web Services), a graphical processing unit (GPU), etc., can be used to implement the disclosed techniques.

The subsystems shown in FIG. 9 are interconnected via a system bus 75. Additional subsystems such as a printer 74, keyboard 78, storage device(s) 79, monitor 76, which is coupled to display adapter 82, and others are shown. Peripherals and input/output (I/O) devices, which couple to I/O controller 71, can be connected to the computer system by any number of means known in the art such as input/output (I/O) port 77 (e.g., USB, FireWire). For example, I/O port 77 or external interface 81 (e.g. Ethernet, Wi-Fi, etc.) can be used to connect computer system 10 to a wide area network such as the Internet, a mouse input device, or a scanner. The interconnection via system bus 75 allows the central processor 73 to communicate with each subsystem and to control the execution of a plurality of instructions from system memory 72 or the storage device(s) 79 (e.g., a fixed disk, such as a hard drive, or optical disk), as well as the exchange of information between subsystems. The system memory 72 and/or the storage device(s) 79 may embody a computer readable medium. Another subsystem is a data collection device 85, such as a camera, microphone, accelerometer, and the like. Any of the data mentioned herein can be output from one component to another component and can be output to the user.

A computer system can include a plurality of the same components or subsystems, e.g., connected together by external interface 81 or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware (e.g. an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, units, circuits, or other means for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover reference to a “first” or a “second” component does not limit the referenced component to a particular location unless expressly stated.

All patents, patent applications, publications, and descriptions mentioned herein are incorporated by reference in their entirety for all purposes. None is admitted to be prior art.

Attached to this description is an Appendix that includes additional information regarding certain embodiments. Other terms used in the Appendix also may not (yet) be terms commonly used in the industry.

1-30. (canceled)

31. A computer-implemented method comprising:
determining an initial ancestral origin estimate of each of a plurality of segments of an input genomic sequence of a subject, wherein each of the segments comprises a sequence of single nucleotide polymorphisms (SNP) values; and
for each of the segments:
identifying one or more neighboring segments of the segment;
determining the initial ancestral origin estimate of each of the one or more neighboring segments; and
processing the initial ancestral origin estimate of the segment and the one or more neighboring segments to obtain a prediction result for an ancestral origin of the segment.

32. The method of claim 31, further comprising:
storing a machine learning model trained based on segments representing training genomic sequences that have known ancestral origins; and
receiving data representing the input genomic sequence of the subject, the input genomic sequence covering the segments including a plurality of SNP sites of a genome of the subject, wherein each of the segments comprises the sequence of SNP values at the SNP sites, each of the SNP values specifying a variant at the SNP site,
wherein the trained machine learning model and the data are used to determine the initial ancestral origin estimate of each of the segments of the input genomic sequence of the subject.

33. The method of claim 32, wherein:
the machine learning model comprises one or more predictor units,
determining the initial ancestral origin estimate of each of the segments comprises inputting a sequence of SNP values of a different segment of the segments to the one or more predictor units to generate the initial ancestral origin estimate of the segment,
the initial ancestral origin estimate comprises one of a classification output or a coordinates output,
the classification output indicates an ancestral origin category, out of a plurality of candidate ancestral origin categories, of the segment input to the predictor unit, and the coordinates output includes coordinates indicative of the ancestral origin or a breed of the segment.

34. The method of claim 33, wherein the coordinates comprise geographical coordinates of a locale of the ancestral origin in a physical space.

35. The method of claim 33, wherein:
the coordinates comprise breed coordinates, and
subjects of different breeds have different breed coordinates generated from genomic sequences of the subjects of the different breeds.

36. The method of claim 35, wherein:
the breed coordinates are defined in a multi-dimensional space being defined by dimensions obtained from a dimension reduction operation on an encoding of SNP sites, and
the machine learning model is trained using vectors representing genomic sequences of reference subjects and reference breed coordinates obtained from performing the dimension reduction operation on the vectors.

37. The method of claim 31 wherein determining the initial ancestral origin estimate of each of the segments comprises:
determining, for each of a plurality of candidate ancestral origins, a probability of the segment being classified into the candidate ancestral origin; and
selecting the candidate ancestral origin having the highest probability as the ancestral origin of the segment.

38. Apparatus comprising:
one or more processors configured to perform:
determining an initial ancestral origin estimate of each of a plurality of segments of an input genomic sequence of a subject, wherein each of the segments comprises a sequence of single nucleotide polymorphisms (SNP) values; and
for each of the segments:
identifying one or more neighboring segments of the segment;
determining the initial ancestral origin estimate of each of the one or more neighboring segments; and
processing the initial ancestral origin estimate of the segment and the one or more neighboring segments to obtain a prediction result for an ancestral origin of the segment.

39. The apparatus of claim 38 further comprising:
memory for storing a machine learning model trained based on segments representing training genomic sequences that have known ancestral origins; and
an input for receiving data representing the input genomic sequence of the subject, the input genomic sequence covering the segments including a plurality of SNP sites of a genome of the subject, wherein each of the segments comprises the sequence of SNP values at the SNP sites, each of the SNP values specifying a variant at the SNP site,
wherein the trained machine learning model and the data are used to determine the initial ancestral origin estimate of each of the segments of the input genomic sequence of the subject.

40. The apparatus of claim 39 wherein:
the machine learning model comprises one or more predictor units,
determining the initial ancestral origin estimate of each of the segments comprises inputting a sequence of SNP values of a different segment of the segments to the one or more predictor units to generate the initial ancestral origin estimate of the segment,
the initial ancestral origin estimate comprises one of a classification output or a coordinates output,
the classification output indicates an ancestral origin category, out of a plurality of candidate ancestral origin categories, of the segment input to the predictor unit, and
the coordinates output includes coordinates indicative of the ancestral origin or a breed of the segment.

41. The apparatus of claim 40, wherein the coordinates comprise geographical coordinates of a locale of the ancestral origin in a physical space.

42. The apparatus of claim 40, wherein:
the coordinates comprise breed coordinates, and
subjects of different breeds have different breed coordinates generated from genomic sequences of the subjects of the different breeds.

43. The apparatus of claim 42, wherein:
the breed coordinates are defined in a multi-dimensional space being defined by dimensions obtained from a dimension reduction operation on an encoding of SNP sites, and
the machine learning model is trained using vectors representing genomic sequences of reference subjects and reference breed coordinates obtained from performing the dimension reduction operation on the vectors.

44. The apparatus of claim 38, wherein determining the initial ancestral origin estimate of each of the segments comprises:
determining, for each of a plurality of candidate ancestral origins, a probability of the segment being classified into the candidate ancestral origin; and
selecting the candidate ancestral origin having the highest probability as the ancestral origin of the segment.

45. A non-transitory computer-readable storage medium for storing code that, when executed by a computer, causes performance of operations comprising:
determining an initial ancestral origin estimate of each of a plurality of segments of an input genomic sequence of a subject, wherein each of the segments comprises a sequence of single nucleotide polymorphisms (SNP) values; and
for each of the segments:
identifying one or more neighboring segments of the segment;
determining the initial ancestral origin estimate of each of the one or more neighboring segments; and
processing the initial ancestral origin estimate of the segment and the one or more neighboring segments to obtain a prediction result for an ancestral origin of the segment.

46. The non-transitory computer-readable storage medium of claim 45, wherein the initial ancestral origin estimates of the segments are determined using a machine learning model trained based on segments representing training genomic sequences that have known ancestral origins and data representing the input genomic sequence of the subject, the input genomic sequence covering the segments including a plurality of SNP sites of a genome of the subject, each of the segments comprising the sequence of SNP values at the SNP sites, each of the SNP values specifying a variant at the SNP site.

47. The non-transitory computer-readable storage medium of claim 46, wherein:
the machine learning model comprises one or more predictor units,
determining the initial ancestral origin estimate of each of the segments comprises inputting a sequence of SNP values of a different segment of the segments to the one or more predictor units to generate the initial ancestral origin estimate of the segment,
the initial ancestral origin estimate comprises one of a classification output or a coordinates output,
the classification output indicates an ancestral origin category, out of a plurality of candidate ancestral origin categories, of the segment input to the predictor unit, and
the coordinates output includes coordinates indicative of the ancestral origin or a breed of the segment.

48. The non-transitory computer-readable storage medium of claim 47, wherein the coordinates comprise geographical coordinates of a locale of the ancestral origin in a physical space.

49. The non-transitory computer-readable storage medium of claim 47, wherein:
the coordinates comprise breed coordinates, and
subjects of different breeds have different breed coordinates generated from genomic sequences of the subjects of the different breeds.

50. The non-transitory computer-readable storage medium of claim 49, wherein:
the breed coordinates are defined in a multi-dimensional space being defined by dimensions obtained from a dimension reduction operation on an encoding of SNP sites, and
the machine learning model is trained using vectors representing genomic sequences of reference subjects and reference breed coordinates obtained from performing the dimension reduction operation on the vectors.
